(19) United States
(12) Patent Application Publication
(10) Pub. No.: US 2004/0106146 A1
(43) Pub. Date: Jun. 3, 2004

(54) Immunochemical methods

(76) Inventors: Timo Palosuo, Espoo (FI); Tytti Karkkainen, Espoo (FI); Vladimir Ovod, Tampere (FI); Markku Kulomaa, Siivikkala (FI); Kai Krohn, Salmentaka (FI); Pekka Sillanaukee, Tampere (FI); Nisse Kalkkinen, Espoo (FI); Timo Reunala, Kangasala (FI); Kristiina Turjanmaa, Helsinki (FI); Harri Alenius, Helsinki (FI)

Correspondence Address: NIXON & VANDERHYE, PC, 1100 N GLEBE ROAD, 8TH FLOOR, ARLINGTON, VA, 22201-4714, US

(21) Appl. No.: 10/203,883

(22) PCT Filed: Feb. 15, 2001
(86) PCT No.: PCT/FI01/00148

(30) Foreign Application Priority Data
Feb. 15, 2000 (FI) 20000326
Oct. 9, 2000 (FI) 20002225

(51) Int. Cl. G01N 33/53; G01N 33/537; G01N 33/543

(52) U.S. Cl. 435/7.1; 435/7.92

(57) ABSTRACT

The present invention relates to immunochemical analysis methods, which are useful in the detection of natural rubber latex allergens or specific antibodies against them. Specifically, the present invention relates to immunochemical analysis methods, which are useful in the detection the possible residual content of allergenic proteins or peptides derived from natural rubber latex in a finished product made of or containing natural rubber latex. Additionally, the present invention relates to immunochemical analysis methods, specifically, in the in vitro detection and analysis of specific immunoglobulin E (IgE) and/or G₄ (IgG₄) antibodies against latex allegens in a patient suspected of suffering from latex allergy and in in vivo diagnosis of latex allergy. The present invention further relates to preparations and specific immunochemical test kits useful in such methods.

### FIELD OF THE INVENTION

The present invention relates to immunochemical analysis methods, which are useful in the detection of natural rubber latex allergens or specific antibodies against them. Specifically, the present invention relates to immunochemical analysis methods, which are useful in the detection the possible residual content of allergenic proteins or peptides derived from natural rubber latex in a finished product made of or containing natural rubber latex. Additionally, the present invention relates to immunochemical analysis methods which are useful in the diagnosis of latex allergy, specifically in the in vitro detection and analysis of specific immunoglobulin E (IgE) and/or G₄ (IgG₄) antibodies against latex allegens in a patient suspected of suffering from latex allergy and in in vivo diagnosis of latex allergy. The present invention further relates to preparations and specific immunochemical test kits useful in such methods.

### BACKGROUND OF THE INVENTION

Products containing natural rubber latex (NRL) from the rubber tree Hevea brasiliensis are widely used due to the economical price and advantageous processing properties of natural rubber. However, adverse reactions against a number of allergenic proteins contained in the NRL are well known and documented, and in the past years allergy to NRL has become a major occupational problem especially among health care and dental professionals. NRL-containing surgical and protection gloves and various medical devices (like catheters, tubes, masks, etc.) contribute to the major portion of these adverse reactions, and even the glove powder may contain NRL proteins, absorbed from the NRL onto the powder particles, and thus cause both sensitisation and adverse reactions. In healthcare, the NRL-based medical devices exhibit a potential source of sensitisation not only to the personnel but also to the patients undergoing an examination or a surgery. In addition, workers in other professions where protection gloves are used, such as kitchen workers or greenhouse workers, or workers in rubber industry, may become sensitised to allergenic NRL proteins, and they also may be subjected to airborne NRL proteins. Finally, even the general population becomes in a daily contact with diverse NRL-containing products, such as household gloves, condoms and balloons, manufactured by the dipping procedure, and also with tubes, tires, erasers and like.

Although natural rubber latex gloves have been used in medicine and industry for over 100 years, latex allergy as a disease entity has come to the attention of the medical and scientific community not until about 10-15 years ago. Currently, latex allergy is recognised as a serious world wide health problem: a significant proportion of health care workers (3-15%) and approximately 1% of the whole population have become sensitised to natural rubber latex [Turjanmaa, K. et al., Allergy 51 (1996) 593-602; Liss, G. M. and Sussman, G. L., Am. J. Ind. Med. 35 (1999) 196-200; Poley Jr, G. E. and Slater, J. E., J. Allergy Clin. Immunol. 105 (2000) 1054-1062]. Importantly, due to an increasing need for protection against HIV, hepatitis B, and other infectious agents, the frequency to exposure and, subsequently, the number of sensitisations are going up.

When susceptible individuals are exposed to NRL-containing products, both irritant and allergic reactions may occur. The potentially most serious adverse reaction is an IgE-mediated allergy, which can manifest as local or systemic symptoms. Localised symptoms include mild contact urticaria at the site of contact, e.g. in the hands, but the skin reaction may spread and become generalised. Systematic symptoms comprise allergic rhinoconjunctivis and asthma, the anaphylactic reaction being the most serious manifestation, and the seriousness of condition is accented by the fact that the first sign of sensitisation can manifest as a life-threatening reaction.

Latex gloves are considered as the principal sensitisers and as the largest single source of exposure, and they are responsible for the majority of the immediate Type I or IgE-mediated hypersensitivity reactions to latex. Approximately one fifth of people in western populations are atopic (i.e. inclined to produce large amounts of IgE antibodies) and thus generally at increased risk for developing latex allergy. Well-defined risk groups include health care workers and other glove-using personnel and children with spina bifida or other congenital malformations and histories of multiple surgical operations at an early age.

Latex allergens are proteins or polypeptides eluting from the manufactured products upon contact with skin, mucous membranes or other tissues. According to the current allergen nomenclature system maintained by the International Union of Immunological Societities (IUIS) under the WHO, 11 latex allergens, which have been characterised at the primary structure level and are contained in the official allergen list, are named as set forth in Table 1.

TABLE 1
Nomenclature of natural latex allergens

| | | Molecular |
|---|---|---|
| Nomenclature | Name | weight, kD |
| Hev b 1 | Rubber Elongation Factor (REF) | 14.6 |
| Hev b 2 | β-1,3-glucanase | 36 |
| Hev b 3 | Rubber particle associated protein | 23 |
| Hev b 4 | Microhelix protein complex | 50-57* |
| | | 100-110** |
| Hev b 5 | Acidic 16 kD protein | 16 |
| Hev b 6.01 | Prohevein | 20 |
| Hev b 6.02 | Hevein | 4.7 |
| Hev b 6.03 | Prohevein C-domain | 14 |
| Hev b 7 | Patatin-like protein | 46 |
| Hev b 8 | Profilin | 15.7 |
| Hev b 9 | Latex enolase | 47.6 |
| Hev b 10 | Latex Mn-SOD | 22.9 |

′*reduced form;
**non-reduced form

The complete primary structure of these latex allergens with nomenclature assignation (except for Hev b 4) is currently known, and the genes encoding these proteins have been cloned and the corresponding recombinant proteins produced. At present, four of these allergens (i.e. Hev b 1, Hev b 3, Hev b 5 and Hev b 6.02) have unequivocally been demonstrated in manufactured latex products. Nel and Gujuluva [Annals of Allergy, Asthma and Immunology 81 (1998) 388-398] disclose the percentage of allergic responses to Hev b 1, Hev b 3, Hev b 5 and Hev b 6.02/Hev b 6.01 (hevein/prohevein) in different patient groups, as set forth in Table 2, wherein LSP denotes latex sensitive people, HCW denotes health care workers, and SBP denotes Spina bifida patients.

TABLE 2
Major latex allergens

| | Rate of allergic response in | |
|---|---|---|
| Antigen | different patient groups | |
| Hevein/prohevein | 75-83% | in SP |
| | 75% | in HCW |
| | 27% | in SBP |
| Hev b 1 | 22% | in LSP |
| | 51-100% | in SBP |
| | 52% | in HCW |
| Hev b 3 | 76% | in SBP |
| Hev b 5 | 95% | in HCW |
| | 52% | in LSP |

Other latex allergens are likely to be important in the commonly occurring allergen cross-reactions. In all, over 50 IgE-binding proteins or polypeptides have been detected in NRL [Alenius, H. et al, J. Lab. Clin. Med., 123 (1994) 712-720].

However, in spite of the recent progress in identifying and characterisation of various latex allergens including the production of polyclonal and monoclonal antibodies against these allergens, no suitable commercial tests for the measurement of specific residual allergen content in NRL-containing products are available. In particular, no method for a simultaneous measurement of latex allergens of clinical relevance is available.

The serious allergic reactions caused by latex products and the commonness of the condition have prompted the international and national health authorities to seek for means to solve the problems. An easy solution would be the use of alternative synthetic materials. However, due to the advantageous properties and reasonable price of natural rubber latex, and also due to its importance to the economy of many countries, the synthetic materials are not able to compete with NRL as a source material. On the other hand, although a large proportion of the proteins shown to be allergenic could be removed from the NRL by the modern technology, they cannot be totally removed moved from the NRL, since these proteins contribute both to the manufacturing properties, i.e. to the barrier properties and like, of the NRL and to the elasticity and the comfort of use of the final NRL-products.

Consequently, a specific goal of the regulatory authorities, i.e. the requirement that the manufacturers produce and sell only allergen-free or low-allergen medical or industrial latex products to stop the sensitisation, cannot easily be achieved. A pertinent approach would be to develop and offer for general use a specific and reliable test for measuring and monitoring the amounts of latex allergens in NRL-containing products, the test being easy enough to be performed by both the users and the producers of these products.

According to the obvious need, the FDA in the USA and the CEN in Europe have recently adopted the measurement of the total protein by a modified Lowry method as the first means to monitor the purported allergen levels of medical gloves made of natural rubber latex. However, there is as yet no agreement on limits for acceptable protein levels in medical gloves. Additionally and importantly, the total protein and allergen concentrations do not correlate satisfactorily with each other. Nevertheless, since no suitable commercial tests for the measurement of specific residual allergen content in NRL-containing products are available, insensitive and non-illustrating total protein measurements are currently in use.

The total protein of NRL-containing products is currently being measured with the conventional Lowry method [Lowry, O. H. et al., J. Biol. Chem. 193 (1951), 265-275] or with the modified Lowry as endorsed by the FDA (ASTM D 5712-95) and the CEN, or with the LEAP or Latex ELISA for Antigenic Protein (Guthrie Research Institute, Sayre, Pa., USA), which is the only commercially available test kit for the measurement latex antigens. In these methods, the detection limit of the total protein varies from about 0.06-9.3 mg/ml, and these methods suffer from several disadvantages. For instance, fairly large amounts of the sample are required, the presence of interfering substances, such as surfactants, detergents or non-latex derived proteins, may cause false results, and the methods are time-consuming to perform. Another disadvantage is that the method is based on the absorbance of certain amino acid residues that have to be present in the peptide chain in an average manner. However, the greatest disadvantage of these methods is the inherent fact that they measure the total protein or antigen content, not the particular allergen content, in a sample.

Similarly, no balanced preparations exist for the diagnosis of IgE-dependent latex allergy and different commercial skin prick test preparations as well as different serological tests have varying latex allergen content. Usually they rely on crude extracts from liquid latex of Hevea brasiliensis. These extracts contain, in addition to the desired latex allergens, a non-defined mixture of other proteins, macromolecules and chemical substances, which may interfere the tests, e.g., by causing false positive or negative reactions, by inhibiting the adsorption of the desired latex allergen to the solid phase or by covering the reaction of a latex allergen with the specific IgE antibody. A serious drawback of a latex extract is the fact that it may, in a batch-wise fashion, contain different amounts of relevant latex allergens, resulting in over- or under-representation of certain latex allergens in terms of others in a SPT preparation or on a solid phase and thereby in false negative/positive reactions. Some allergens may be totally absent from the preparation used in the skin test or immobilised on the solid phase. Moreover, when extracts are prepared for skin prick tests, their reactivity can be adjusted only by diluting which results in non-reactive levels of those allergens which originally are present in small amounts. Often, as indicated in Table 2, people may be allergic to a particular allergen and not to others. If this particular allergen is absent from the preparation, latex allergy will not be detected. False positive reactions may also occur because of food allergies, which may be completely unrelated to latex allergy. On the other hand, if reactions against allergens present in such small amounts are desired, the concentrations of abundant allergens and other components may become risky.

In the diagnosis of IgE-dependent latex allergy, the patient's clinical history is usually verified by an in vivo skin prick test (SPT) on a patient's forearm and/or by an in vitro serological test from a patient's blood sample. Occasionally challenge tests, in which an allergen preparation is inhaled, are also performed.

In the skin prick test, a small drop of an allergen solution is placed on the forearm of the patient and a needle prick is made with a standardised needle through the drop into the skin. In a positive test, a wheal and flare reaction is observed within 15 minutes, and the reaction is compared to a reaction obtained with a 10 mg/ml histamine hydrochloride solution (positive control) and an isotonic NaCl-solution (negative control). The SPT is generally considered as the “gold standard” in demonstrating sensitisation to latex and diagnosing latex allergy. The sensitivity of the SPT has been reported to vary from 54 to 96 % (depending on the test reagents) and the specificity between 95-100 % [Ebo, D. G. et al., J. Allergy Clin. Immunol. 100 (1997) 618-623].

In vitro tests detect specific IgE antibodies against a specific allergen in a patient's serum. Allergens are immobilised on a solid phase, such as a paper disc, a test tube, a bead or a well of a microtiter plate. Allergen-specific IgE, if present in the serum sample, binds to the allergen, and the allergen-IgE-complex is detected with a labelled antibody against human IgE. The relative sensitivity compared to the SPT, when tested with sera from patient having positive clinical history and positive STP to NRL, varies from appr. 73 to 95% and the relative specificity from appr. 33-97.2 % [Ebo, D. G. et al., J. Allergy Clin. Immunol. 100 (1997) 618-623; Kim, K. T. and Sadafi, G. S., J. Allergy Clin. Immunol. 97 (1996) 1188-1191; Ownby, D. R. et al., Ann. Allergy Asthma Immunol 84 (2000) 193-196; Hamilton, R. G., et al., J. Allergy Clin. Immunol. 103 (1999) 925-930]. However, when non-selected blood donors are tested with a commercial serological test, specific IgE to NRL are observed in 5.4 to 7.6% [Saxon, A. et al., All. Allergy Asthma Immunol. 84 (2000) 199-206; Ownby, D. R. et al., J. Allergy Clin. Immunol. 97 (1996) 1188-1191; Merret, T. G. et al., Clin. Exp. Allergy 29 (1999) 1572-1578].

Both in vivo and in vitro tests are dependent of the quality of the allergen preparation used: if the allergen preparation lacks one or more relevant allergens, neither the skin prick test nor the detection of specific IgE antibodies from the serum sample is complete and correct.

Due to the lack of suitable commercial preparations and/or negative experiences obtained therewith, “home-made” latex allergen preparations are widely used. Such home-made preparations are prepared by extracting the latex allergens for instance from surgical gloves into a suitable buffer. Not surprisingly, the quality and the allergen content vary considerably from batch to batch and there is no guarantee that all latex allergens are present in such an allergen extract.

Further, the SPT requires competent and trained personnel to ensure the reliability and the validity of the test. The SPT also exhibits a potential, although minimal risk of an anaphylactic reaction.

An in vitro serological test with a specificity and sensitivity of the SPT would be optimal in view of the above. In presently used latex allergy tests, however, the specificity, sensitivity, reproducibility, and validity are unsatisfactory, regardless of the source of a latex extract, since no balanced preparations exist.

The serious allergic reactions caused by latex products and the prevalence of latex allergy call for improved methods and reagents for a reliable detection of NRL-allergens and for a reliable diagnosis of NRL-allergy. An inherent requirement of safety and reliability, regardless of whether the detection of a possible presence of latex allegens in a finished NRL-containing product or a skin prick test, a challenge test, or a measurement of specific IgE antibodies is concerned, is that all latex allergens having clinical relevance are detected or present in the latex allergen preparation. Thus, improved test preparations, methods and reagents, which unequivocally comprise or detect all clinically relevant latex allergens, are urgently needed.

EP patent application 0 704 457 suggests and claims three latex allergens isolated by chromatographic methods from natural rubber latex in a substantially purified form, namely Hev b 4, Hev b 2 and Hev b 3, and their use in the determination of specific IgE or in an assay for the qualitative and/or quantitative determination of the levels of allergens of natural rubber latex present in latex containing material. Only one of the proteins disclosed, i.e. Hev b 3, has unequivocally been shown to be present in manufactured latex products. Furthermore, each of the allergens is measured separately.

Recently a concept of component-resolved diagnostics (CRD) has been introduced to assess a patient's individual pattern of sensitisation for immunotherapy [for review see Valenta et al., Clin Exp Allergy 29 (1999) 896-904]. By CRD the development and course of an allergic disease can be monitored and traced back to individual allergens of the material causing the allergy, and with recombinant allergens it is possible to determine to which individual allergens the patient has become sensitised. Again, each allergen is tested separately. For diagnostic purposes the use of such a large panel of individual recombinant allergens is uneconomical, laborious and time consuming to perform and thus not suitable for routine analysis.

A simultaneous measurement of the residual content of latex allergens of clinical relevance in a finished NRL-product is essential in view of the fact that the adverse reaction to various latex allergens contained in a product differs from an individual to another, i.e. the allergic individuals are allergic to different latex allergens. Thus even among individuals with a diagnosed latex allergy, a natural latex product may cause a fatal reaction in one allergic individual but be totally harmless to another allergic individual, who on the other hand may get a severe reaction from another product containing another specific allergen. This means that a conventional method, which specifically detects only one latex allergen, fails in reliability and security from the user's respect. A simultaneous measurement of at least two clinically relevant latex allergens, let alone a simultaneous measurement of all clinically relevant latex allergens, significantly increases the reliability, security and usefulness of a method.

Similarly, a simultaneous and reliable detection of all specific IgE or IgG₄ antibodies against at least two, preferably all latex allergens of clinical relevance in a biological sample from a patient suspected of suffering from latex allergy is crucial for a reliable latex allergy diagnosis.

Thus there is an urgent need for reliable, reproducible, sensitive and specific tests for the simultaneous measurement of the residual content of latex allergens of clinical relevance in NRL-containing products and for a reliable diagnosis of latex allergy. In addition to a test, which can be used by skilled laboratory personnel, there is also an urgent need for rapid and simple tests suitable for the latex-allergic or to risk group-belonging users of NRL-containing products or for workers in the latex industry.

The present invention provides a solution to overcome the disadvantages and drawbacks described above and offers new reliable approaches in the art.

In one aspect of the invention, the invention provides reliable quantitative analysis methods for the simultaneous measurement of the residual content of natural rubber latex allergens of clinical relevance in a finished product made of or containing natural rubber latex. Such reliable quantitative analysis methods would allow for instance the regulatory authorities in heath care to control and monitor the marketed latex-based products. Similarly, such quantitative analysis methods would allow the manufacturers of rubber products to specifically and reliably control the content of latex allergens in their products and to produce consumer-safe products.

In another aspect of the invention, the invention provides reliable guantitative/semiquantitative diagnostic methods for the detection of specific IgE/IgG₄ to at least two, preferably all natural rubber latex allergens of clinical relevance in a patient sample. Such semiquantitative analysis methods would increase the specificity and reliability of latex allergy diagnosis. Furthermore, these methods should be applicable to any standard immunological method platform as well as to a dedicated instrument.

In a further aspect of the invention, the invention provides rapid qualitative/semiquantitative screening methods for the simultaneous measurement of the residual content of natural rubber latex allergens of clinical relevance in a finished product made of or containing natural rubber or for the detection of specific IgE and/or IgG₄ antibodies to natural rubber latex allergens of clinical relevance in a patient's sample. Such qualitative/semiquantitative screening methods would allow latex allergic or atopic or other users quickly and reliably to analyse the product to be used. Thereby these screening methods would benefit the users of the product and/or, in the case of, e.g., a surgery or a medical examination, guarantee the safety of the product to the patient or they would allow a quick and reliable diagnosis of, for instance, a patient undergoing a surgery or a medical examination, and thereby add to the safety of such procedures. Additionally, these methods would allow the diagnosis of NRL allergy even in situations, where laboratory services are not available.

In still another aspect of the invention, the invention provides means for practising the methods of the invention, i.e. test kits suitable for use in the quantitative or qualitative/semiquantitative analysis methods of the invention.

In a still further aspect of the invention, the invention provides compositions of clinically relevant latex allergens containing fixed, predetermined concentrations of each allergen. Such compositions containing at least two, preferably all clinically relevant latex allergens are useful in skin prick test or in a challenge test or for the preparation of reagents for in vitro serological tests. Because the amounts used in the test can be adjusted to any desired level, the reliability and the safety of skin prick tests and challenge tests increase. A risk of anaphylactic reactions may also decrease.

The present invention provides highly specific and sensitive immunochemical methods for the simultaneous measurement of the residual content of natural rubber latex allergens of clinical relevance in a finished product made of or containing natural rubber latex and for the detection of specific IgE and/or IgG₄ against clinically relevant latex allergens in a patient sample. The decrease in the number of false positive and false negative results demonstrates the increased specificity and sensitivity. Specifically, false positive reactions, which are due to shared or similar allergenic epitopes between some latex allergens and certain fruits and foods, decrease or may totally disappear.

### SHORT DESCRIPTION OF THE INVENTION

It was unexpectedly found that it is possible to develop novel immunochemical methods for a simultaneous measurement of clinically relevant latex allergens and for measurement of specific IgE and/or IgG₄ against clinically relevant latex allergens, taking advantage of known immunological principles and methods. Accordingly, the present invention provides immunochemical analysis and diagnostic methods which are based on the use of recombinant natural rubber latex allergens, and in particular, on the use of an advantageous and specific combination of individual, preferably recombinant latex allergens or a fusion protein thereof with a suitable fusion partner or functionally equivalent fragments or aggregates thereof, preferably in desired adjustable amounts, and on the specific antibodies reacting therewith.

In the present context, the terms “latex allergens of clinical relevance” and “clinically relevant latex allergens” refer to those natural rubber latex allergens which are of clinical relevance due to the fact that they can retain their allergenic properties through the manufacturing processes and are detectable in finished products made of or containing natural rubber latex. These latex allergens include at present Hev b 1, Hev b 3, Hev b 5, and Hev b 6.02, all of which unequivocally have been demonstrated in finished NRL-containing products. However, the scope of the invention is also intended to cover the detection of all those so far non-identified latex allergens, which raise IgE-mediated immunological response in a susceptible individual and which retain their allergenicity in a manufactured product, i.e., which have clinical relevance.

In one aspect of the invention, the present invention relates to immunochemical methods for simultaneous demonstrating and quantifying and/or qualitatively/semiquantitatively demonstrating at least two, and preferably, all specific latex allergens having clinical relevance from manufactured rubber products, such as various medical devices, especially gloves.

According to this aspect, the present invention specifically relates to homologous and heterologous immunochemical methods comprising

(a) binding at least two specific latex allergens having clinical relevance with primary antibodies, said antibodies being (mono)specific polyclonal antibodies, which are directed against respective clinically relevant latex allergens or their functionally equivalent breakdown fragments, sub-units or aggregates or their fusion proteins with a suitable partner, or monoclonal antibodies, which are directed against specific epitopes of the respective clinically relevant latex allergens or their functionally equivalent breakdown fragments, sub-units or aggregates or their fusion proteins with a suitable partner, said primary antibodies being optionally bound to a solid phase, and

(b) detecting the bound allergens with secondary antibodies, said antibodies being other (mono)specific polyclonal antibodies, which are directed against respective clinically relevant latex allergens or their functionally equivalent breakdown fragments, sub-units or aggregates or their fusion proteins with a suitable partner, or monoclonal antibodies, which are directed against other specific epitopes of the respective latex allergens having clinical relevance or their functionally equivalent breakdown fragments, sub-units or aggregates or their fusion proteins with a suitable partner, said secondary antibodies optionally being suitably labelled and/or optionally being bound to a solid phase.

The present invention further relates to homologous or heterologous immunochemical methods comprising

(a) binding at least two specific latex allergens having clinical relevance with primary antibodies, said antibodies being (mono)specific polyclonal antibodies, which are directed against respective clinically relevant latex allergens or their functionally equivalent breakdown fragments, sub-units or aggregates or their fusion proteins with a suitable partner, or monoclonal antibodies, which are directed against specific epitopes of the respective clinically relevant latex allergens or their functionally equivalent breakdown fragments, sub-units or aggregates or their fusion proteins with a suitable partner, said primary antibodies optionally being bound to a solid phase, and

(b) detecting the bound allergens with secondary polyclonal antibodies raised against a mixture of the respective latex allergens having clinical relevance or their functionally equivalent breakdown fragments, sub-units or aggregates or their fusion proteins with suitable partners, said secondary antibodies optionally being suitably labelled and/or optionally being bound to a solid phase.

The present invention further relates to homologous or heterologous immunochemical methods comprising

(a) binding at least two specific latex allergens having clinical relevance with primary and secondary antibodies, said primary antibodies being (mono)specific polyclonal antibodies, which are directed against respective clinically relevant latex allergens or their functionally equivalent breakdown fragments, sub-units or aggregates or their fusion proteins with a suitable partner, or monoclonal antibodies, which are directed against specific epitopes of the respective clinically relevant latex allergens or their functionally equivalent breakdown fragments, sub-units or aggregates or their fusion proteins with a suitable partner, and said secondary antibodies being other (mono)specific polyclonal antibodies, which are directed against respective clinically relevant latex allergens or their functionally equivalent breakdown fragments, sub-units or aggregates or their fusion proteins with a suitable partner, or monoclonal antibodies, which are directed against other specific epitopes of the respective latex allergens having clinical relevance or their functionally equivalent breakdown fragments, sub-units or aggregates or their fusion proteins with a suitable partner, said primary and secondary antibodies being bound to a solid phase and optionally being suitably labelled for detection, and

(b) detecting the bound allergens by a suitable detection means, such as colorimetry, turbidometry, nephelometry or fluorometry.

In a preferred embodiment, monoclonal antibodies are used as primary and/or secondary antibodies. In another preferred embodiment, monoclonal antibodies are used as primary antibodies and polyclonal antibodies are used as secondary antibodies.

In a specially preferred embodiment of the invention, four clinically relevant latex allergens, i.e. Hev b 1, Hev b 3, Hev b 5, and Hev b 6.02, are detected with monoclonal antibodies against the respective four clinically relevant latex allergens or their functionally equivalent breakdown fragments, sub-units or aggregates or their fusion proteins with a suitable partner.

In another preferred embodiment of the invention, as applicable, all clinically relevant latex allergens are detected with monoclonal antibodies against the respective clinically relevant latex allergens or their functionally equivalent breakdown fragments, sub-units or aggregates or their fusion proteins with a suitable partner.

In a further preferred embodiment of the invention, the primary antibodies are bound to a solid phase and the secondary antibodies are suitably labelled.

In a further preferred embodiment of the invention, the primary and secondary antibodies are bound to a solid phase, and turbidometry is used for the detection.

According to another aspect of the invention, the present invention relates to an immunochemical method for detecting the presence or absence of specific IgE and/or IgG₄ antibodies against latex allergens of clinical relevance, the method comprising contacting a specimen, which is obtained from a patient suspected of suffering from latex allergy, with said clinically relevant latex allergens and detecting the immunological complex thus formed. In this aspect, the present invention specifically relates to an immunochemical method for detecting the presence or absence of specific IgE and/or IgG₄ antibodies against at least two, preferably four, natural latex allergens of clinical relevance, whereby the specimen is contacted with at least two, preferably four, natural rubber latex allergens of clinical relevance, preferably recombinant natural latex allergens or fusion proteins thereof with a suitable fusion partner, or functionally equivalent fragments or aggregates thereof, the amounts of said allergens being suitably adjusted to ensure high specificity and sensitivity and thus valid results.

In a preferred embodiment of the invention the specimen is contacted with at least Hev b 6.02 and any one or all of Hev b 1, Hev b 3 and Hev b 5. Preferably, recombinant forms of these allergens or fusion proteins thereof with a suitable fusion partner, or functionally equivalent fragments or aggregates thereof, are used.

In a preferred embodiment of the invention the specimen is contacted with preferably recombinant Hev b 6.02 and preferably recombinant Hev b 5 or fusion proteins thereof with a suitable fusion partner or functionally equivalent fragments or aggregates thereof.

In another preferred embodiment of the invention the specimen is contacted with preferably recombinant Hev b 3 and preferably recombinant Hev b 1 or fusion proteins thereof with a suitable fusion partner or functionally equivalent fragments or aggregates thereof.

In a specifically preferred embodiment of the invention the specimen is contacted with preferably recombinant Hev b 1, Hev b 3, Hev b 5 and Hev b 6.02 or fusion proteins thereof with a suitable fusion partner or functionally equivalent fragments or aggregates thereof.

The present invention further relates to a rapid screening method for the detection of the presence or absence of specific IgE and/or IgG₄ against latex allergens of clinical relevance, the method comprising contacting a specimen, which is obtained from a patient suspected of suffering from latex allergy, with clinically relevant latex allergens and detecting the immunological complex thus formed. In particular, the specimen is contacted with at least two, preferably four natural rubber latex allergens of clinical relevance, preferably recombinant natural rubber latex allergens or fusion proteins thereof with a suitable fusion partner or functionally equivalent fragments or aggregates thereof, whereby the amounts of said allergens are suitably adjusted to reliably and rapidly differentiate between NRL allergy positive and negative patients, for instance in emergency situations, to avoid severe reactions caused by NRL allergy.

The present invention further relates to test kits for performing the immunochemicl methods of the invention. According to one aspect of the invention, the present invention relates to test kits for determining at least two specific latex allergens having clinical relevance in a product made of or containing natural rubber latex, the test kit comprising

(a) a first set of at least two antibodies, the antibodies being (mono)specific polyclonal antibodies, which are directed against respective specific latex allergens having clinical relevance or their functionally equivalent breakdown fragments, sub-units or aggregates or their fusion proteins with a suitable partner, or monoclonal antibodies, which are directed against specific epitopes of the respective specific latex allergens having clinical relevance or their functionally equivalent breakdown fragments, sub-units or aggregates or their fusion proteins with a suitable partner, said first antibodies optionally being bound to a solid phase,

(2a) a second set of at least two antibodies, the antibodies being (mono)specific polyclonal antibodies, which are directed against other respective specific latex allergens having clinical relevance or their functionally equivalent breakdown fragments, sub-units or aggregates or their fusion proteins with a suitable partner, or monoclonal antibodies, which are directed against another epitope of the respective specific latex allergens having clinical relevance or their functionally equivalent breakdown fragments, sub-units or aggregates or their fusion proteins with a suitable partner, said antibodies optionally being suitably labelled and/or bound to a solid phase, or

(2b) polyclonal antibodies raised against a mixture of at least two specific latex allergens having clinical relevance or their functionally equivalent breakdown fragments, sub-units or aggregates or their fusion proteins with suitable partners, said antibodies being optionally suitably labelled and/or bound to a solid phase,

(3) a reagent or reagents needed for the detection, and

(4) the standards needed for the quantification of the specific latex allergens having clinical relevance.

Additionally, the test kit may optionally contain (5) a reagent or reagents for the elution of the latex allergens having clinical relevance from a sample suspected to contain such allergens, and/or (6) a reagent or reagents for the possible washing steps, and/or (7) a latex allergen control reagent or reagents.

In a preferred embodiment of the invention, the test kit contains the reagents necessary for the detection of four clinically relevant latex allergens, i.e. for the detection of Hev b 1, Hev b 3, Hev b 5, and Hev b 6.02. In another preferred embodiment, the test kit of the invention contains the reagents necessary for the detection of all clinically relevant latex allergens, as applicable.

Additionally, the present invention relates to a composition for skin prick tests or challenge tests containing predetermined amounts of natural rubber latex allergens of clinical relevance, preferably recombinant natural rubber latex allergens or fusion proteins thereof with a suitable fusion partner or functionally equivalent fragments or aggregates thereof in a suitable formulation.

In a preferred embodiment of the invention the composition contains at least preferably recombinant Hev b 6.02 and any of preferably recombinant Hev b 1, Hev b 3 and Hev b 5 or fusion proteins thereof with a suitable fusion partner or functionally equivalent fragments or aggregates thereof. In the most preferred embodiment of the invention the composition contains preferably recombinant Hev b 1, Hev b 3, Hev b 5 and Hev b 6.02 or fusion proteins thereof with a suitable fusion partner or functionally equivalent fragments or aggregates thereof.

The present invention further relates to the use of preferably recombinant Hev b 1, Hev b 3, Hev b 5 and Hev b 6.02 or fusion proteins thereof in the diagnosis of NRL allergy and in the detection of the residual allergen content in a NRP product.

Although the principle of these immunochemical methods is known, they have not previously been utilised in the latex allergy field for the simultaneous detection and measurement of the residual content of clinically relevant latex allergens in a finished product containing natural rubber latex. The simultaneous use of selected purified and standardised recombinant latex allergens and specific antibodies reacting therewith in a specifically defined combination makes the methods of the invention highly specific and of exquisite sensitivity, and capable of detecting the specific latex allergens, or epitopes thereof, in the nanogram or μM range.

Neither is the simultaneous use of selected purified and/or recombinant natural rubber latex allergens in a specifically defined, balanced combination in the diagnosis of latex allergy described previously. This novel approach makes the methods of the invention, unlike the known methods, highly specific and of exquisite sensitivity, and capable of detecting the specific IgE and/or IgG₄ against latex allergens, or epitopes thereof, in a semiquantitative or, if desired, quantitative manner.

The methods of the invention are suitable for rubber manufacturers to be used in quality control and product monitoring in order to assist the attempts to produce and offer for sale only low-allergen or allergen-free products, for health authorities providing them a means to execute surveillance and market analyses, and for healthcare personnel and other users for testing the NRL-containing material. Similarly, the methods of the invention are suitable for reliable diagnosis of latex allergy.

### SHORT DESCRIPTION OF THE DRAWINGS

FIG. 1 shows the standard curve of recombinant avidin-Hev b 6.02 fusion protein and Hev b 6.02 content of glove samples on a microtiter plate coated with a monoclonal antibody raised against recombinant avidin-Hev b 6.02 fusion protein.

FIG. 2 shows a standard curve of recombinant MBP-Hev b5 fusion protein and Hev b 5 content of glove samples on a microtiter plate coated with monoclonal antibody against recombinant MBP-Hev b5.

FIG. 3 shows the comparison of results from 14 patient serum samples analysed on microtiter plates coated with recombinant Hev b 1, Hev b 3, Hev b 5 and Hev b 6.02 either individually or in combination. FIG. 1a. Microtiter plates coated with Hev b 1, Hev b 3, Hev b 1+Hev b 3 and Hev b 1+Hev b 3+Hev b 5+Hev b 6.02. FIG. 1b. Microtiter plates coated with Hev b 5, Hev b 6.02, Hev b 5+Hev b 6.02 and Hev b 1+Hev b 3+Hev b 5+Hev b 6.02.

FIG. 4 is a graphical picture of results obtained by the method of the invention.

FIG. 5 shows the construction (a) and the result (b) of a rapid screening test of the invention.

### DETAILED DESCRIPTION OF THE INVENTION

The invention is based on the simultaneous use specific natural rubber latex allergens, preferably recombinant natural rubber latex allergens of clinical relevance, or fusion proteins thereof with a suitable fusion partner or functionally equivalent fragments or aggregates thereof. When applied to the detection and identification and/or quantification of clinically important latex allergens in NRL-containing products with a single assay, these allergens are used together with (mono)specific polyclonal and/or monoclonal antibodies directed against these allergens in a homologous or heterologous immunochemcal method of the invention. When used for the detection of specific IgE and/or IgG₄ antibodies against these allergens in a patient's sample an increased sensitivity and specificity is achieved. If desired, even a quantitative measurement of specific antibodies possible. When used for skin prick test or challenge test it is ensured that the test solutions contain predetermined amounts of the desired NRL allergens.

The specific natural latex allergens of clinical relevance useful in the present invention can be prepared by conventional purification methods or, as preferred, by recombinant technology. Methods of the purification of natural latex allergens are well known by persons skilled in the art. For instance, native Hev b 1 and Hev b 3 can be purified as described by Alenius, H. et al. [Int. Arch. Allergy Immunol. 109 (1996) 362-368], native Hev b 5 can be purified as described by Akasawa, A. et al., [J. Biol. Chem. 271 (1996) 25389-25393] and native Hev b 6.02 can be purified as described by Alenius, H. et al. [J. Immunol. 156 (1996) 1618-1625]. Methods for the production of recombinant allergens are also known to persons skilled in the art. The production using the recombinant technology offers some advantages, for instance in terms of the ease of purification and coating properties brought by the fusion partners.

For preparation of the recombinant latex allergens or their fusion proteins a reference is made to Examples 2 to 6. Any known established expression method for preparing recombinant proteins can, however, be used. Thus, prokaryotic expression systems, such as E. coli, Bacillus subtilis and like, and other eukaryotic expression systems, e.g., yeast, fungi and mammalian cells, are equally suitable for the purpose. With both prokaryotic and eukaryotic expression systems, the recombinant latex allergens can be produced as individual proteins or as fusion proteins with proteins conventionally used as a fusion partner, such as avidin, streptavidin or maltose binding protein (MBP) as well as HIS-tag, glutathione-S-transferase or other available isolation and purification tags. The bacterial expression systems are well characterised and inexpensive, and they produce high quantities of the desired proteins. Although most of the post-translational modifications are missing from the produced recombinant proteins, they can be used as highly specific immunogens in the antibody production. Eukaryotic expression systems, on the other hand, produce proteins with post-translational modifications.

The fusion protein approach provides means for an easy chromatographic isolation and purification of fusion proteins. For instance MBP-fusion proteins can be purified by affinity chromatography on an amylose column and avidin-fusion proteins on a biotin-agarose, 2-iminobiotin-agarose column orother biotin derivatives coupled to an insoluble matrix. The recombinant latex allergens can then be cleaved from the fusion protein with a suitable enzyme or by a chemical cleavage without impairing the immunological or other biological properties of the allergen. For instance, the MBP-fusion proteins can be cleaved by Factor Xa protease and the avidin-fusion proteins by enteroxinase or thrombin.

For a definitive identification, all the recombinant latex allergens were carefully analysed by standard methods, such as SDS-electrophoresis, immunoblot analysis, N-terminal sequencing and (MALDI-TOF) mass spectrometry. In addition to these structural analyses, the immunological properties of the recombinant allergenic NRL-derived proteins were analysed by standard methods, such as by an inhibition ELISA assay using previously characterized sera from patients with NRL allergy. The immunological properties were also compared to the corresponding purified native rubber tree proteins. Functional properties such as interaction with other molecules are, when applicable, analysed by the optical biosensor technology using instruments such as BiaCore or IaSys. In all cases, the recombinantly produced latex allergens had structural and functional properties very similar or identical to the corresponding native proteins.

The recombinant latex allergens having clinical relevance and being capable to maintain their allergenicity in the manufacturing process, with and without a fusion partner, create the basis to produce specific panels of monoclonal antibodies and (mono)specific polyclonal antibodies. For the purposes of some embodiments of the invention, polyclonal and monoclonal antibodies were prepared against the above mentioned different recombinant latex allergens or their fusion proteins, and their isotypes were determined using standard procedures well known in the art. In this respect, a reference is made to Examples 7and 8.

Generally, the antibodies useful in the methods of the present invention can be either mono- or polyspecific polyclonal antibodies, monoclonal antibodies or recombinant single chain monoclonal antibodies or antigen-binding fragments thereof that are specific to latex allergens having clinical relevance or their functionally equivalent breakdown fragments, sub-units or aggregates. For use in a homologous immunochemical method of the invention (the allergen binding antibodies or the primary antibodies originate from the same animal species), the monoclonal antibodies have to be specific to the different epitopes. For use in the heterologous immunochemical method of the invention (the allergen binding antibodies originate from the different animal species), the monoclonal antibodies (with consequent utilizing anti-murine subclass-specific labelled monoclonal or polyclonal antibodies) have to belong to the different murine Ig isotypes. In cases where the binding antibody is a monoclonal antibody and the detecting antibody or the secondary antibody is a labelled polyclonal antibody, the murine isotype is not critical.

Preferably, the antibodies useful in the present invention for the binding of the latex allergens are monoclonal antibodies or monospecific polyclonal antibodies or functional fragments of these that are specific to certain molecular epitopes present in the latex allergens having clinical relevance. The most advantageous antibodies for the purpose of binding the latex allergens are monoclonal antibodies. Any types of monoclonal antibodies can be used but those belonging to the murine IgG subclasses (isotypes) are preferable, since they can be effectively purified by immunoaffinity chromatography.

Preferably, the antibodies useful in the present invention for the detection of the latex allergens are monoclonal antibodies thereof or monospecific polyclonal antibodies or functional fragments of these, which are specific to certain different molecular epitopes present in the latex allergens having clinical relevance. In one preferred embodiment of the invention, polyclonal antibodies raised against a mixture of recombinant latex allergens having clinical relevance or their fusion proteins are employed due to the simplicity of use.

To enable the simultaneous measurement of the clinically relevant latex allergens, the specific antibodies used as primary or binding antibodies or secondary or detecting antibodies in the immunochemical methods of the invention were mixed in predetermined amounts. These predetermined amounts and dilutions were determined on the basis of separate analyses of each of the individual recombinant allergen or a fusion protein thereof, by e.g., the ELISA method. Accordingly, primary and secondary antibody mixtures useful in the invention are mixtures of monoclonal antibodies directed against recombinant Hev b 6.02 and Hev b 5, or against recombinant Hev b 6.02 and Hev b 3, or against recombinant Hev b 6.02 and recombinant Hev b 1, or against any other suitable combination of two recombinant clinically relevant latex allergens or their fusion proteins, wherein the primary and secondary antibodies are directed to different epitopes of respective latex allergens. Alternatively, primary and secondary antibody mixtures useful in the invention are mixtures of monoclonal antibodies against all clinically relevant recombinant latex allergens, such as recombinant forms of Hev b 6.02, Hev b 5, Hev b 3 and Hev b 1 or their fusion proteins, wherein the primary and secondary antibodies are directed to different epitopes of respective latex allergens. Alternatively, the binding antibodies can be used separately, i.e., they can be coupled to different parts of the solid phase.

The detecting antibodies as well as the detecting monoclonal or polyclonal anti-human IgE and/or IgG₄ antibodies or fragments thereof can be labelled with any conventionally used label or marker. Accordingly, the labels useful in the methods of the present invention include an enzyme label, a biotin-avidin system, a fluorescent, fosforescent or luminescent label, a radioactive label or a chromophore, a gold label or any other conventionally used label. Preferred labels are enzyme labels, such as horse radish peroxidase. Alternatively, the secondary antibodies can be non-labelled and tertiary labelled antibodies are used for the detection.

The immunological methods of the invention may utilise any known solid phase technique currently used for antigen detection of for the detection of specific IgE and IgG₄ antibodies provided that the requirements set forth in the claims are fulfilled, such as the principles of ELISAs (Enzyme Linked ImmunoSorbent Test), the RAST (RadioAllergo Sorbent Test), fluorescent immunoallergo sorbent test or FEIA, turbidometry, nephelometry, competitive tests, time-resolved fluorometry and like (see Immunoassay, Diamandis, E. P. and Christopoulus, T. K., Eds. (1997), AACC Press, USA).

Suitable materials for the solid phase are synthetic materials, such as polystyrene, polyvinylchloride, polyamide and the other conventionally used synthetic polymers, as well as natural polymers, such as cellulose, derivatised natural polymers, such as cellulose acetate and nitrocellulose, paper and glass.

The solid phase can be in a form of a microtiter plate, a tube, a stick or a bead as conventionally used in the art. Also paper strips, plates or membranes form a suitable solid phase useful in the invention. Preferred solid phases are microtiter plates, tubes, sticks, paper, synthetic or nitrocellulose membrane strips. The most preferred materials are microtiter plates and, for the rapid qualitative/semiquantitative methods, synthetic polymeric material or nitrocellulose membranes.

The latex allergens are attached to the solid phase by any conventional means. Usually incubating the antigen solution in a suitable buffer, such as 50-200 mM PBS, phosphate buffer pH 6.0-8.0 and like, on the solid phase for a predetermined time, for instance 2 to 24 hours at room temperature, provides a reproducible attachment on microtiter plates. Similarily, the primary and/or secondary antibodies are attached to the solid phase by any conventional means. Usually incubating the antibody solution in a suitable buffer, such as PBS, phosphate buffer and like, on the solid phase for a predetermined time provides reproducible attachment. Nitrocellulose can be coated simply by dispensing or spraying the allergen solution in a suitable buffer, such as those described above, as a line or figure on the membrane. Paper strips can be coated by covalent chemical coupling using glutaraldehyde or cyanogen bromide as coupling agent by methods known in the art.

The solid phase can contain separate areas, e.g. wells in a microtiter plate or zones on a polystyrene stick or on a nitrocellulose membrane, which contain a suitable antibody or allergen mixture. Alternatively, the individual recombinant latex allergens or their fusion proteins or the specific antibodies thereto can be coupled to different wells or zones on the solid phase. For instance, microtiter plates can be constructed containing separate rows of wells coupled with specific binding antibodies to each latex allergen of clinical relevance.

The latex allergens are reasonably well water-soluble. Thus, generally the solvent for preparing latex allergens can be any suitable aqueous buffer, such as a phosphate buffer, phosphate buffered saline (PBS), acetate buffer, TRIS buffer and like, having a molarity in the range of about 10 to 200 mM and the pH in the range of about 6 to 9 optionally containing detergents. A preferred buffer is 100 mM PBS. For certain purposes, for instance in skin prick test solutions to stabilise the allergens or to increase the viscosity of the solution, additives, such as sugars or glycerol, may be added.

The sample to be tested can be any product suspected to contain latex allergens, such as a medical or protective glove, a tube, a catheter, a mask and like. It can also be a solution or a sample of a specimen from a certain manufacturing step. Alternatively, the specimen to be tested can be serum, plasma, total blood saliva, lacrimal fluid, or any other sample normally used in a measurement of specific IgE or IgG₄, undiluted or diluted in a suitable buffer.

The test kit according to one aspect of the present invention contains the reagents needed for performing the immunological methods of the invention. Thus in one embodiment of the invention the test kit contains a first set, preferably a mixture, of (mono)specific polyclonal or monoclonal antibodies, preferably monoclonal antibodies, bound to a solid phase, such as one of those mentioned above. The antibodies of the first set are directed against one epitope of each respective specific latex allergen having clinical relevance or its functionally equivalent breakdown fragment, sub-unit or aggregate or its fusion protein with suitable partner. In another embodiment of the invention the test kit contains a solid phase, such as one of those mentioned above, on which the combination of clinically relevant latex allergens, preferably recombinant allergens or their functionally equivalent breakdown fragments, sub-units or aggregates or their fusion proteins with suitable partner, has been attached.

In one presently preferred embodiment, the test kit contains a solid phase which is coupled with a mixture, which comprises monoclonal antibodies against at least two latex allergens having clinical relevance, for instance, against recombinant Hev b 5 and recombinant Hev b 6.02 or their suitable recombinant fusion proteins, recombinant Hev b 6.02 and recombinant Hev b 1 or their suitable recombinant fusion proteins, recombinant Hev b 6.02 and recombinant Hev b 3 or their suitable recombinant fusion proteins, or any combination of two clinically relevant latex allergens or their suitable fusion proteins. In a specifically preferred embodiment, the test kit contains a solid phase, which is coupled with a mixture, which comprises monoclonal antibodies against all latex allergens known to have clinical relevance, i.e. against recombinant Hev b 1, Hev b 3, Hev b 5 and Hev b 6.02 or their suitable recombinant fusion proteins, or with two mixtures, each comprising a different pair of monoclonal antibodies against two clinically relevant monoclonal antibodies.

The test kit contains additionally a second set, preferably a mixture, of (mono)specific polyclonal or monoclonal antibodies, preferably monoclonal antibodies, each of which is directed against another epitope of each respective specific latex allergens having clinical relevance or its functionally equivalent breakdown fragment, sub-unit or aggregate or its fusion protein with suitable partner. In a presently preferred embodiment, the mixture comprises antibodies against at least two latex allergens having clinical relevance, for instance, against recombinant Hev b 5 and recombinant Hev b 6.02 or their suitable recombinant fusion proteins, recombinant Hev b 6.02 and recombinant Hev b 1 or their suitable recombinant fusion proteins, recombinant Hev b 6.02 and recombinant Hev b 3 or their suitable recombinant fusion proteins, or any combination of two clinically relevant latex allergens or their suitable fusion proteins. In a specifically preferred embodiment, the solid phase is coupled with a mixture, which comprises monoclonal antibodies against all latex allergens known to have clinical relevance, i.e. the mixture comprises monoclonal antibodies against at least recombinant Hev b 1, Hev b 3, Hev b 5 and Hev b 6.02 or their suitable recombinant fusion proteins. These antibodies are optionally suitably labelled with a suitable label, such as one of those mentioned above, and/or optionally bound to a solid phase as described above, to enable the detection.

Alternatively, the test kit contains, to enable the detection of the latex allergens, labelled polyclonal antibodies raised against a mixture of the respective specific latex allergens having clinical relevance or their functionally equivalent breakdown fragments, sub-units or aggregates or their fusion proteins with suitable partners. In a presently preferred embodiment, the test kit comprises antibodies against at least two latex allergens having clinical relevance, for instance, against recombinant Hev b 5 and recombinant Hev b 6.02 or their suitable recombinant fusion proteins, recombinant Hev b 6.02 and recombinant Hev b 1 or their suitable recombinant fusion proteins, recombinant Hev b 6.02 and recombinant Hev b 3 or their suitable recombinant fusion proteins, or any combination of two clinically relevant latex allergens or their suitable fusion proteins. In a specifically preferred embodiment, the test kit contains polyclonal antibodies raised against a mixture of at least recombinant Hev b 1, Hev b 3, Hev b 5 and Hev b 6.02 or their suitable recombinant fusion proteins.

Alternatively, the specific antibodies to individual recombinant latex allergens or their fusion proteins can be coupled to different wells or zones on the solid phase. For instance, microtiter plates can be constructed containing separate rows of wells coupled with specific binding antibodies to each latex allergen of clinical relevance.

According another aspect of the invention, the test kit contains a solid phase which is coupled with a combination of at least two clinically relevant NRL allergens, such as Hev b 6.02 and any of the group comprising Hev b 1, Hev b 3 and Hev b 5 or their suitable recombinant forms or their fusion proteins. In a presently preferred embodiment the test kit contains a solid phase which is coupled with Hev b 1, Hev b 3, Hev b 5 and Hev b 6.02 or their suitable recombinant forms or their fusion proteins.

The test kit contains additionally polyclonal or monoclonal antibodies against human IgE or IgG₄, preferably monoclonal antibodies suitably labelled with a marker, such as one of those mentioned above, to enable the detection.

The test kit further contains a reagent or reagents needed for the detection, such as a substrate for the label enzyme, a quenching reagent, a colour-developing reagent, a pH indicator, where the reaction changes the pH, a reagent changing the ionic strength, a detergent and like. Additionally, the test kit of the invention may contain a positive and/or negative control sample. In an embodiment using non-labelled secondary antibody, a labelled tertiary antibody, such as an HRP-labelled anti-IgG antibody, is included in the test kit of the invention.

Standards or calibrators needed for the quantification/semiquantification/ qualification of the specific latex allergens having clinical relevance are also included in the test kits of the invention. In a presently preferred embodiment, the standard include two recombinant allergens of clinical relevance, such as recombinant Hev b 5 and recombinant Hev b 6.02 or their suitable recombinant fusion proteins, recombinant Hev b 6.02 and recombinant Hev b 1 or their suitable recombinant fusion proteins, recombinant Hev b 6.02 and recombinant Hev b 3 or their suitable recombinant fusion proteins, or any combination of two clinically relevant latex allergens or their suitable fusion proteins. In a specifically preferred embodiment, the test kit of the present invention includes at least recombinant Hev b 1, Hev b 3, Hev b 5 and Hev b 6.02 or their suitable recombinant fusion proteins as standards, preferably all in one vial. Alternatively, the calibrators can be human serum pools in different dilutions to give a negative, borderline, weak positive, positive or strong positive reaction.

Additionally, the test kit may optionally contain a reagent or reagents for the elution of the latex allergens having clinical relevance from a sample suspected to contain such allergens. Such reagents include, e.g., PBS, a phosphate buffer, an acetate buffer or any other buffer mentioned above. Similarly, the washing solutions or concentrates thereof, such as PBS-0.05% Tween, may be included.

The methods of the invention afford unique specificity and sensitivity. When sera from patients with clinical history to NRL and positive skin prick reactions to various NRL allergens were tested with a method of the invention using a microtiter plate coated with equal amounts of recombinant Hev b 1, Hev b 3, Hev b 5 and Hev b 6.02, all sera (100%) gave a positive reaction (Table 4). The three control sera from volunteers with no NRL allergy were all negative. Weakly positively reacting sera (patients 9 and 11 in Table 3) reacted more strongly in the method of the invention. In contrast to this, when the same sera were tested using microtiter plates coated with a field latex extract (i.e. an extract prepared from latex milk and containing 12 mg/ml of protein by the Lowry method), 2/16 samples (patients 4 and 9 in Table 3) were negative, and one sample gave a borderline result (patient 12).

When these patient sera were tested with a method of the invention using a microtiter plate coated with equal amounts of recombinant Hev b 5 and Hev b 6.02, 15/16 (94%) sera were positive. The only non-reactive serum was from a patient sensitive only to Hev b 1.

When these sera were tested with a method of the invention using a microtiter plate coated with equal amounts of recombinant Hev b 1 and Hev b 3, only those sera which were clearly positive for either Hev b 1 or Hev b 3 gave a positive reaction. Thus only the correct patients were recognised.

As is evident for the persons skilled in the art, the basic idea of the invention can be practised in many different ways at present and in the future. Thus the invention and its embodiments are not intended to be limited by the following examples but these are given only as illustrative examples of the invention.

### EXAMPLE 1

### Preparation of Native Hev b 1, Hev b 3, Hev b 5 and Hev b 6.02

Native Hev b1 was purified essentially as described by Alenius, H. et al. [Int. Arch. Allergy Immunol. 109 (1996) 362-368]. Native Hev b 3 was purified essentially following the principles described by Alenius. H. et al. (supra) for Hev b1. Native Hev b 5 was purified essentially as described by Akasawa, A. et al., [J. Biol. Chem. 271 (1996) 25389-25393]. Native Hev b 6.02 was purified essentially as described by Alenius, H. et al. [J. Immunol. 156 (1996) 1618-1625].

### EXAMPLE 2

### Preparation of Recombinant Hev b 6.02

For the preparation of the recombinant Hev b 6.02 or hevein (sequence id. no. 11), the hevein coding fragment was amplified by PCR from a pIIProhev plasmid essentially as described by Airienne, K. J. et al. [ Prot. Express. Purif. 9 (1997) 100-108]. PCR Prohev6 [(5′-TA TGT GGA TCC GAC GAC GAC GAC AAA GAG CAA TGT GGT CGG CAA GCA-3′(sequence id. no. 3); BamHI site underlined; enterokinase site in bold; hevein sequence in italics] was used as the forward primer and Prohev2 [5′-AAC ACA AGC TT C TTA GTC TTT GCA ATT GCT TTG GC-3′ (sequence id. no. 4); HindIII site underlined; stop codon in bold; hevein sequence in italics] as the reverse primer. The PCR was performed as described by Airenne. K. J. and Kulomaa, M. S. [Gene 167 (1995) 63-68]. After digestion with Bg/II+HindIII (Promega, Madison, Wis., USA), the PCR product was applies to a 1.5% preparative agarose gel. The fragment, which was of the expected size, was recovered from the gel as described by Heery et al. [TIG 6 (1990) 173] and purified further with the Magic™ DNA Clean-up System (Promega). The purified fragment was subcloned into the pFastBAC1-donor plasmid (GIBCO BRL, Gaithersburg, Md., USA), and recombinant viruses containing the Hev b 6.02 sequence were then generated using the Bac-TO-Bac Baculovirus Expression System (GIBCO BRL, Gaithersburg, Md., USA) according to the manufacturer's instructions.

Alternatively, recombinant Hev b 6.02 can be obtained by the enzymatic cleavage of the protein from a fusion protein (cf. Example 3)

### EXAMPLE 3

### Preparation of Recombinant Avidin-Hev b 6.02 Fusion Protein

To create recombinant viruses containing both the Hev b 6.02 and the avidin coding regions, the amplified fragment containing Hev b 6.02 sequence and obtained as described in Example 1 was subcloned into pFastBAC1-based vector pbacAVs+C, which contains a secretion-compatible form of recombinant avidin, essentially as described by Airenne, K. J. et al. [Prot. Express. Purif. 17 (1999) 139-145].

Subsequently, for the preparation of avidin- 6.02 fusion protein, ˜5 ×10⁷ Sf9 cells (ATCC CRL 1711) in SF-900 II SFM serum-free culture medium depleted of biotin and Pluronic F68 (GIBCO BRL, Gaithersburg, Md., USA; 041-94100A) were seeded to a final volume of 25 ml in a 250 ml Erlenmeyer flask. Recombinant viruses were added to give a multiplicity of infection of 2 Pfu/cell. After three days of incubation at 27° C. in a shaker at 125 rpm, the cells were pelleted by centrifugation (1000×g, 22° C., 5 min) and the pellet was frozen under liquid nitrogen and stored at −70° C.

The crude avidin-Hev b 6.02 fusion protein was purified and characterised as decribed in Airenne, K. J. et al. (1999, supra). The purified fusion protein was shown to have equivalent biotin-binding properties to native eggwhite avidin. The hevein portion was also found to have functional and structural properties similar or identical to those of native hevein.

### EXAMPLE 4

### Preparation of Recombinant MBP-Hev b 1 Fusion Protein and Recombinant Hev b 1

For the preparation of the recombinant Hev b 1 or REFor rubber elongation factor (sequence id. no. 5), the Hev b 1 coding fragment was amplified by PCR from a REF-pMALc-2-plasmid. The PCR forward primer was 5′-ATGGCTGAAGACGAAGACAACCAA-3′(Hev b 1 sequence in italics; sequence id. no. 6), and the reverse primer 5′-GATAT-CAAGCTTTCAATTCTCTCCATAAAACACCTTA-3′, (HindIII site underlined; Hev b 1 sequence in italics; sequence id. no. 7). The PCR was performed as described by Airenne. K. J. and Kulomaa, M. S. [Gene 167 (1995) 63-68]. After digestion with HindIII (Promega, Madison, Wis., USA), the PCR product was applied to a 1.5% preparative agarose gel. The fragment, which was of the expected size, was recovered from the gel as described by Heery et al. [TIG 6 (1990) 173] and purified further with ethanol precipitation. The purified fragment was subcloned into the pMAL-c2-plasmid that had been digested by XmnI+HindIII enzymes. The sequence of the recombinant plasmid was checked by an automated DNA sequencing instrument.

When this recombinant plasmid was introduced into the E coli JM109host cells according to the manufacturer's instructions (New England BioLabs Inc., Hitchim, Herfordshire, UK), a MBP-Hev b 1 fusion protein with a Factor Xa cleavage site in between the MBP-tag and the allergen was produced. The purification of the MBP-Hev b 1 fusion protein was performed according to a method which was designed for purification of a soluble MBP-fusion protein expressed into the cytoplasm from pMAL-c2 vector (New England BioLabs Inc., Hitchim, Herfordshire, UK). The isolation was based on affinity chromatography on an Amylose column.

Cleavage of the MBP-tag from Hev b 1 protein can be performed by Factor Xa protease to obtain recombinant Hev b 1.

### EXAMPLE 5

### Preparation of Recombinant MBP-Hev b 3 Fusion Protein and Recombinant Hev b 3

For the preparation of the recombinant Hev b 3 or rubber particle associated protein (sequence id. no. 8), the Hev b 3 coding fragment was amplified by PCR from pBS+SRPP-plasmid. The PCR forward primer was 5′-ATGGCTGAAGAGGTGGAGGA-3′(Hev b 3 sequence in italics; sequence id. no. 9), and the reverse primer 5′-GATATCAAGCTTTTATGATGCCTCATCTCCAA-3′(HindIII site underlined; Hev b 3 sequence in italics; sequence id. no. 10). The PCR was performed as described by Airenne. K. J. and Kulomaa, M. S. [Gene 167 (1995) 63-68]. After digestion with HindIII (Promega, Madison, Wis., USA), the PCR product was applied to a 1.5% preparative agarose gel. The fragment, which was of the expected size, was recovered from the gel as described by Heery et al. [TIG 6 (1990) 173] and purified further with ethanol precipitation. The purified fragment was subcloned into the pMAL-c2-plasmid that had been digested by XmnI+HindIII enzymes. The sequence of the recombinant plasmid was checked by an automated DNA sequencing instrument. When this recombinant plasmid was introduced into the E coli JM109 host cells according to the manufacturer's instructions (New England BioLabs Inc., Hitchim, Herfordshire, UK), a MBP-Hev b 3 fusion protein with a Factor Xa cleavage site in between the MBP-tag and the allergen was produced. Purification of the MBP-Hev b 3 fusion protein was performed according to a method which was designed for purification of a soluble MBP-fusion protein expressed into the cytoplasm from pMAL-c2 vector (New England BioLabs Inc., Hitchim, Herfordshire, UK). The isolation was based on affinity chromatography on an Amylose column.

The cleavage of the MBP-tag from Hev b 3 protein can be performed by Factor Xa protease to obtain recombinant Hev b 3.

### EXAMPLE 6

### Preparation of Recombinant MBP-Hev b 5 Fusion Protein

For the preparation of the recombinant Hev b 5 or acidic 16 kD protein (sequence id. no. 12), the Hev b 5 coding fragment was amplified by PCR from a Hevb 5-pMALc-2 plasmid. The PCR forward primer was 5′-ATGGCCAGTGTTGAGGTTGA-3′(Hev b 5 sequence in italics, sequence id. no. 1), and the reverse primer 5′-GATAT-CAAGCTTTTATTCCTCTGTTTTTTC-3′ (HindIII site underlined; sequence id. no. 12). The PCR was performed as described by Airenne. K. J. and Kulomaa, M. S. [Gene 167 (1995) 63-68]. After digestion with HindIII (Promega. Madison, Wis., USA), the PCR product was applied to a 1.5% preparative agarose gel. The fragment, which was of the expected size, was recovered from the gel as described by Heery et al. [TIG 6 (1990) 173] and purified further with ethanol precipitation. The purified fragment was subcloned into the pMAL-c2-plasmid that had been digested by XmnI+HindIII enzymes. The sequence of the recombinant plasmid was checked by an automated DNA-sequencing instrument.

When this recombinant plasmid was introduced into the E coli JM109 host cells according to the manufacturer's instructions (New England BioLabs Inc., Hitchim, Herfordshire, UK) a MBP-Hev b 5 fusion protein with a Factor Xa cleavage site in between the MBP-tag and the allergen was produced. Purification of the MBP-Hev b 5 fusion protein was performed according to a method which was designed for purification of a soluble MBP-fusion protein expressed into the cytoplasm from pMAL-c2 vector (New England BioLabs Inc., Hitchim, Herfordshire, UK). Isolation was based on affinity chromatography on an Amylose column.

Cleavage of the MBP-tag from Hev b 5 protein can be performed by Factor Xa protease.

### EXAMPLE 7

### Preparation of Polyclonal Antibodies Against Recombinant Avidin-Hev b 6.02 fusion protein

The polyclonal antibodies against recombinant avidin-Hev b 6.02 fusion protein were prepared using standard procedures as follows. Rabbits were immunised intramuscularly with 0.05-0.1 mg of recombinant avidin-Hev b 6.02 fusion protein prepared as described in Example 3. In the first immunisations, the antigen was suspended in 0.2 ml of physiological saline and in Complete Freund Adjuvant (1:1) and injected in 4 rabbit body sites (0.05 ml each). In the following boosters Incomplete Freund Adjuvant (1:1) was used. The boosts were given for 3-4 times with the interval 3-4 weeks. The resolution boost was done intravenously. Blood was collected 7-9 days later.

Similar procedures are used for the preparation of polyclonal antibodies against recombinant Hev b 1, Hev b 3, Hev b 5 and Hev b 6.02 or their suitable recombinant fusion proteins or both or mixtures thereof.

### EXAMPLE 8

### Preparation of Monoclonal Antibodies Against Recombinant Hev b6.02 Protein or Recombinant Hev b6.02-Avidin Fusion Protein and Recombinant Hev b5 Protein or Recombinant Hev b5-MBP Fusion Protein

For the generation of monoclonal antibodies, standard strategies and procedures described by Galfre and Millstein [Galfre, G. and Milstein, C., Meth. Enzymol. 73 (1981) pp. 1-46] as well as by Goding, G. W., [Monoclonal antibodies: Principles and Practice (Production and Application of Monoclonal Antibodies in Cell Biology, Biochemistry and Immunology. 1986, pp.1-103, Academic Press, Harcourt Brace Jovanovich Publishers] were used. Recombinant Hev b 6.02 or its recombinant fusion protein with avidin (avidin-Hev b6.02) and recombinant Hev b 5 or its recombinant fusion protein with maltose binding protein (MBP) (MBP-Hev b5), were used as antigens in BALB/c mice. An antigen solution, which contained 15 μg of the respective antigen in 100 μl of 0.1 M phosphate buffered saline (PBS), pH 7.0-7.4, was mixed with 150 μl of Freund's complete adjuvant and injected intraperitoneally to mice. At three weeks' intervals, a booster immunisation with the same dose (15 μg) of the antigen emulsified in Freund's incomplete adjuvant was given intramuscularly in an equal amount in four sites (60 μl/site). Four weeks later, the antigen in a dose of 20 μg in 150 μl of 0.1 M PBS was intravenously administred. Two weeks later, 3 days prior the fusion, an intravenous boost with the same dose was given.

Standard technology of hybridoma production was adapted (see Galfre, G., Milstein, C., supra). Briefly, the splenocytes from immunised mice were prepared and fused with mouse myeloma line Sp2/O-Ag-14 (abbreviation Sp2), which is a total non-producer variant selected from a hybridoma involving fusion of MOPC-21 and BALB/c spleen cells. Polyethylene glycol (PEG), MW 4000, was used as the fusion agent, and the hybridomas were cultivated in a RPMI-1640 medium buffered with HCO₃⁻/CO₂ and containing 10% of fetal calf serum (FCS). Hypoxanthine, aminopterin and thymidine (HAT) were used for the hybridoma selection. The thymocytes from BALB/c mice were used at the early stages of hybridoma growing as well as for recloning.

The hybridomas were primarily screened for the specific monoclonal antibody production by use of an ordinary indirect ELISA with a homologous antigen. Subsequently or in parallel, the hybridomas were screened with fusion protein partner, where a fusion protein had been used as an immunogen, with recombinant purified homologous antigen and natural rubber latex as well glove extracts (GE) (Tables 3 and 4).

Positive clones were further evaluated for the reactivity in Western immunoblotting with recombinant and natural latex proteins. After the specific reactivity had been proved, the isotypes of the monoclonal antibodies were defined using double immunodiffusion in agarose gel or ELISA assays with mouse immunoglobulin class- and subclass-specific non-labelled and labelled antibodies, correspondingly, [Goding, G. W., Monoclonal antibodies: Principles and Practice (Production and Application of Monoclonal antibodies in Cell Biology, Biochemistry and Immunology. 1986, pp.104-107, Academic Press, Harcourt Brace Jovanovich Publishers]. The hybridomas' viability and monoclonal antibody producing potential was evaluated by cloning and recloning of the selected cell lines. These procedures were also aimed to reduce the risk of overgrowth by nonproducer cells, and to ensure that the antibodies are truly monoclonal (Tables 3 and 4).

TABLE 3
Monoclonal antibodies towards H v b 6.02

| Mab | | MAb | Reactivity in ELISA with antigens: | | | | |
|---|---|---|---|---|---|---|---|
| (=cell line) | | production | Av-Hev | Hev b | | | Specific |
| designation | Isotype | stability, % | b 6.02 | 6.02 | NRL* | GE** | epitope |
| Hb6.02-1 | IgG2ₐ | 100 | + | + | + | + | NK*** |
| Hb6.02-2 | IgG1 | 100 | + | + | + | + | NK |

TABLE 4
Monoclonal antibodies towards Hev b 5

| Mab | | MAb | Reactivity in ELISA with antigens: | | | | |
|---|---|---|---|---|---|---|---|
| (=cell line) | | production | MBP-Hev | Hev | | | Specific |
| designation | Isotype | stability, % | b5 | b 5 | NRL* | GE** | peptid |
| Hb5-1 | IgG1 | 85 | + | + | + | + | 101-114 |
| Hb5-2 | IgG1 | 100 | + | + | + | + | NK*** |
| Hb5-3 | IgG1 | 75 | + | + | + | + | NK |
| Hb5-4 | IgG1 | 90 | + | + | + | + | 101-114 |
| Hb5-5 | IgG1 | 100 | + | + | + | + | NK |
| Hb5-7 | IgG1 | 80 | + | + | + | + | 101-114 |
| Hb5-9 | IgG1 | 90 | + | + | + | + | NK |
| Hb5-10 | IgG1 | 100 | + | + | + | + | 101-114 |
| Hb5-11 | IgG2b | 100 | + | + | + | + | 38-51 |

*NRL - Natural rubber latex
**GE - Glove extracts
***NK - Not known

### EXAMPLE 9

### Evaluation of Assay Conditions Useful in a Sandwich-ELISA with Hev b 6.02 Using Latex Glove Extracts

To define and verity the suitable assay conditions, a sandwich-ELISA for Hev b 6.02 was designed. Protection gloves from different manufactures were used as samples.

Monoclonal antibodies prepared as described in Example 8 against the recombinant avidin-Hev b 6.02 fusion protein (clone Hb6-2) 10 μg/ml of 50 mM phosphate buffer, pH 6.0) were applied to 96-well polystyrene microtiter plates (Nunc, Roskilde, Denmark) (150 μl/well) and the plates incubated for 3 hours or overnight at room temperature. The wells were washed with 0,05% PBS-Tween-20, and blocked with 300 μl of 0.5% bovine serum albumin (BSA) in 50 mM phosphate buffer, pH 6.5. Plates were used after removal of blocking solution. The samples (glove extracts) or standards (recombinant Hev b 6.02 as prepared in Example 2) in a volume of 25 μl were added to the appropriate wells followed by addition of 100 μl of assay buffer (50 mM sodium phosphate, 50 mM sodium chloride, 10 mM EDTA, 0.3% BSA, 0.03% Tween-20, pH 7.4). After 30 minutes incubation on a horizontal shaker the plates were washed 4 times with PBS-0.05% Tween-20. One hundred microliters of polyclonal anti-Hev b 6.02 antibodies prepared as described in Example 7 in a dilution of 1:2000 were added and the plates were incubated and washed as in the previous step. One hundred microliters of goat anti-rabbit antibodies labelled with HRP in 1:2000 dilution (Dako P0448) were added, the plates were incubated 15 min and washed as previously. Substrate (ABTS, Kirkegaard-Perry) was added (100 μl) to each well. After 15 minutes of incubation the reaction was stopped with 50 μl of 1% SDS and the absorbances were read at 414 nm (Multiscan, Labsystems).

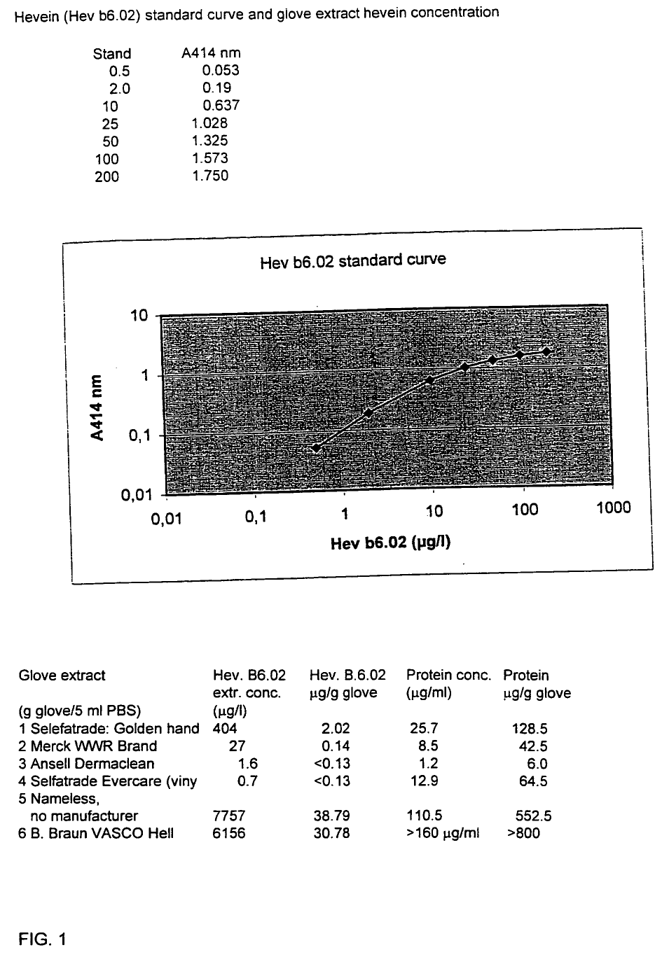

The glove extracts were prepared by cutting the gloves into about 1 cm² pieces, extracting them in PBS (1 g/5 ml PBS) for 2 hrs at room tempererature with occasional swirling. After centrifugation and filtration through a 0.2 μm filter, the extracts were assayed immediately or frozen for later analyses. Concentrations of glove extract Hev b 6.02 were read from the standard curve. The results for the latex glove extracts and the standard curve ranging from 0.5 to 200 μl for the quantification Heb b 6.02 are shown in FIG. 1.

The Hev b 6.02 content in the glove extracts tested varied significantly: the correlation between protein content and Hev b 6.02 content was poor showing that total protein content is indicative for neither allergenicity nor non-allergenicity. Similar results have been obtained using total protein and skin prick tests or RAST inhibition tests.

### EXAMPLE 10

### Evaluation of Assay Conditions Useful in a Sandwich-ELISA with Hev b 5 Using Latex Glove Extracts

To define and verity the suitable assay conditions, a sandwich-ELISA for Hev b 5 was designed. Protection gloves from different manufactures were used as samples.

Monoclonal antibodies prepared as described in Example 8 (clone Hb5-3) against the recombinant avidin-Hev b 5 fusion protein (10 μg/ml in 50 mM sodium phosphate buffer, pH 6.0) 0.1 M sodium phosphate, pH 6.0) were applied to 96-well polystyrene microtiter plates (Nunc. Roskilde, Denmark) (150 μl/well) and incubated overnight at room temperature. The wells were washed and blocked with 300 μl of 0.5% bovine serum albumin (BSA) in 50 mM phosphate buffer pH 6.5. Plates were used after removal of blocking solution.

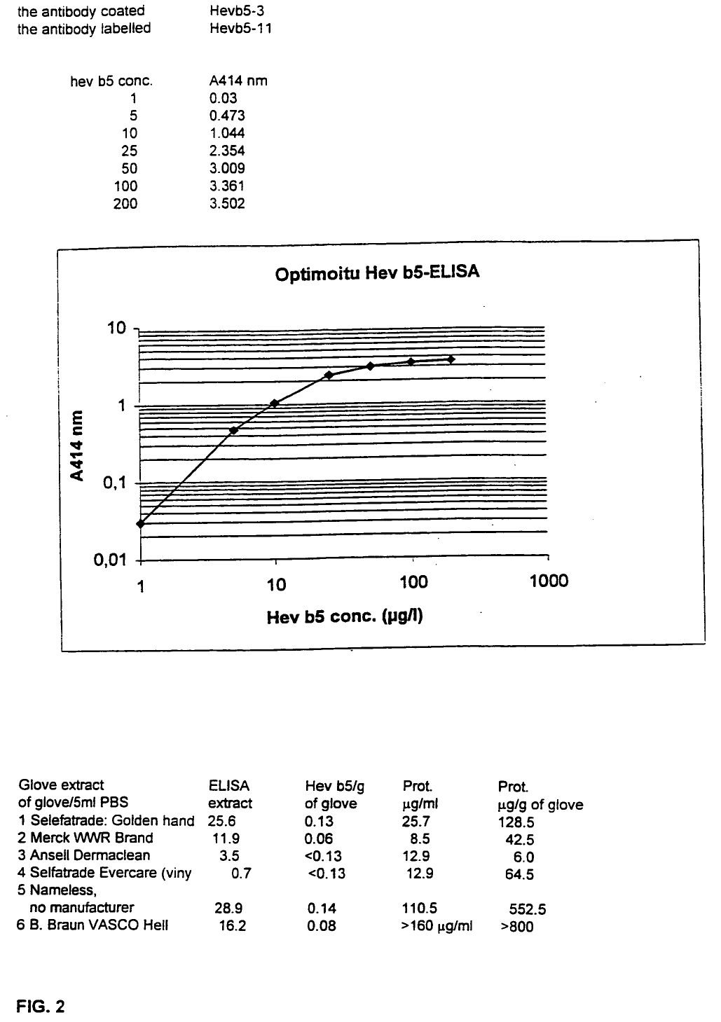

The samples (the glove extracts prepared as described in Example 9) or standards (recombinant Hev b 5 prepared as described in Example 6) in a volume of 50 μl were added to the appropriate wells followed by addition of 100 μl of assay buffer (50 mM sodium phosphate, 50 mM sodium chloride, 10 mM EDTA, 0.3% BSA, 0.03% Tween-20, pH 7.4). After 60 minutes incubation on a horizontal shaker the plates were washed 4 times with PBS-0.05% Tween-20. One hundred microliters of monoclonal anti-Hev b5 (clone Hb5-11) conjugated to HRP in a dilution of 1:200 was added, the plates were incubated for 30 minutes and washed as in the previous step. Substrate (ABTS, Kirkegaard-Perry) was added (100 μl) to each well. After 15 minutes of incubation the reaction was stopped with 50 μl of 1% SDS in distilled water and the absorbances were read at 414 nm (Multiscan, Labsystems). Concentrations of glove extract Hev b5 were read from the standard curve. The results for the latex glove extracts and the standard curve ranging from 1 to 200 μg/l for the quantification Heb b 5 are shown in FIG. 2.

The Hev b 5 content in the glove extracts tested varied significantly. The same amount of Hev b5 can be found in two glove extracts, but the protein concentration was more than five times higher in the other glove extract (no. 1 vs. no. 5). The total protein is indicative for neither allergenicity nor non-allergenicity.

### EXAMPLE 11

### Simultaneous Detection of Hev b5 and Hev b6.02 in Glove Samples

For the preparation of the plates, monoclonal antibodies prepared as described in Example 8 against the recombinant avidin-Hev b 6.02 fusion protein and recombinant MBP-Hev b5 fusion protein, at a predetermined concentration of 1 to 100 μg/ml in 50 mM phosphate buffer, pH 6.0, (150 μl) were applied to 96-well polystyrene microtiter plates (Nunc, Roskilde, Denmark) (150 μl/well), each monoclonal antibody separately to a respective half of the plates, and the plates were incubated for 3 hours or overnight at room temperature. The wells were washed with 0.1 M PBS-Tween-20 (0.05%) and blocked with 300 μl of 0.5% bovine serum albumin (BSA) in 50 mM phosphate buffer, pH 6.5. Plates were used after removal of blocking solution.

The samples (glove extracts) or standards (predetermined amounts of each of recombinant Hev b 6.02 and recombinant Hev b5, in a mixture of a volume of 50 μl were added to the appropriate wells followed by addition of 100 μl of assay buffer (50 mM sodium phosphate, 50 mM sodium chloride, 10 mM EDTA, 0.3% BSA, 0.03% Tween-20, pH 7.4). After 30 minutes incubation on a horizontal shaker the plates were washed 4 times with PBS-0.05% Tween-20, 100 μl of antibodies containing a mixture of monoclonal antibodies against the recombinant avidin-Hev b 6.02 fusion protein and recombinant MBP-Hev b5 fusion protein, in a predetermined ratio, each labelled with horse radish peroxidase (HRP) in predetermined dilutions of 1:100-1:100 000 were added. The plates were incubated for 30 min and washed as previously. Alternatively, polyclonal antibodies raised against a mixture of said fusion proteins can be used. The substrate (ABTS, Kirkegaard-Perry) was added (100 μl) to each well. After 15 minutes of incubation the reaction was stopped with 50 μl of 1% SDS in distilled water and the absorbances were read at 414 nm (Multiscan, Labsystems). The concentrations of Hev b6.02 and Hev b5 in the glove extracts were read from the appropriate standard curves.

### EXAMPLE 12

### Simultaneous Determination of Hev b 6.02 and Hev b 5 on Anti-Hev b 6.02a and Anti-Hev b5 Coated Plates

Simultaneous determination of Hev b 6.02 and Hev b 5 was performed using specific antibodies on the plates, predetermined calibrators and specific labelled antibodies in the same incubation mixture.

For the preparation of the plates, microtiter plates (Maxisorp, Nunc, Denmark) were coated and blocked individually with monoclonal antibodies anti-Hev b 6.02 (clone2) and anti-hev b 5 (clone 4) as described in Example 11.

In the test calibrators containing 0, 2, 7, 40, 100 and 200 μg/l of Hev b 6.02 and 0, 2, 5, 15, 40 and 100 μg/l of Hev b 5 in calibrator buffer (100 mM MES, 50 mM NaCl, 10 mM EDTA, 0.5% Tween-20, 0.5% BSA, 0.1% Proclin 300 pH 6.0 were used as standards. Samples were glove extracts made in PBS as described in Example 9.

In the assay 100 μl of assay buffer (50 mM sodium phosphate, 50 mM sodium chloride, 10 mM EDTA, 0.3% BSA, 0.03% Tween-20, pH 7.4) were pipetted in all wells followed by 25 μl of calibrators or sample. After an incubation of 60 minutes at room temperature on a horizontal shaker (200 rpm) the wells were washed 4 times with PBS-0.05% Tween 20 (Bio Rad Laboratories). One hundred microliters of mixture of antibodies that were labelled with HRP (polyclonal anti-Hev b 6.02 , 1:375, and monoclonal anti-Hev b 5 clone 11, 1:100) in the assay-buffer were added to each well. After an incubation of 30 minutes at room temperature on a horizontal shaker (200 rpm) the wells were washed four times with PBS-0.05% Tween 20 and 100 μl of ABTS substrate (Kirrkegaard-Perry) was added to each well. The plates were incubated at room temperature on a horizontal shaker (200 rpm) and the reaction was stopped with 100 μl of 1% sodium dodecyl sulphate (SDS). Standard curves were plotted for Hev b 6.02 and Hev b 5 and sample values were read from the appropriate curves. Finally the concentrations of both allergens were added together.

TABLE 5
Hev b 6.02 and Hev b 5 in glove extracts

| Calibrator | A414 nm | | A414 nm |
|---|---|---|---|
| Hev b 6.02 | (−blank) | Hev b 5 | (−blank) |
| 2 | 0.019 | 2 | 0.015 |
| 7 | 0.076 | 5 | 0.045 |
| 40 | 0.424 | 15 | 0.252 |
| 100 | 0.981 | 40 | 1.007 |
| | 1.473 | 100 | 2.256 |
| | Hev b 6.02 | Hev b 5 | total |
| | μg/l | μg/l | μg/l |
| Glove 1 | 28 | 34 | 62 |
| Glove 2 | <2 | 8 | 8 |
| Glove 3 | 68 | 20 | 88 |

### EXAMPLE 13

### Simultaneous Detection of Hev b1, Hev b3, Hev b5 and Hev b6.02 In Glove Samples

For the preparation of the plates, monoclonal antibodies as against the recombinant avidin-Hev b 6.02 fusion protein, recombinant MBP-Hev b5 fusion protein, recombinant MBP-Hev b3 fusion protein, recombinant MBP-Hev b1 at a predetermined concentration of 1 to 100 μg/ml in 50 mM phosphate buffer, pH 6.0 were mixed and 150 μl of the mixture was applied to 96-well polystyrene microtiter plates (Nunc, Roskilde, Denmark) (150 μl/well) and incubated for 3 hours or overnight at room temperature. The wells were washed with 0.1 M PBS-Tween-20 (0.05%) and blocked with 300 μl of 0.5% bovine serum albumin (BSA) in 50 mM phosphate buffer, pH 6.5. Plates were used after removal of blocking solution.

The samples (glove extracts) or standards (predetemined amounts of each of recombinant Hev b 6.02, recombinant Hev b5, recombinant Hev b3 and recombinant Hev b1) in a volume of 50 μl were added to the appropriate wells followed by addition of 100 μl of assay buffer (50 mM sodium phosphate, 50 mM sodium chloride. 10 mM EDTA, 0.3% BSA, 0.03% Tween-20, pH 7.4). After 30 minutes incubation on a horizontal shaker the plate was washed 4 times with PBS-0.05% Tween-20, 100 μl of each specific antibody containing polyclonal or monoclonal antibodies against the recombinant avidin-Hev b 6.02 fusion protein, recombinant MBP-Hev b5 fusion protein, recombinant MBP-Hev b3 fusion protein, recombinant MBP-Hev b1 fusion protein labelled with horse radish peroxidase (HRP) in predetermined dilutions of 1:100-1:100 000 were added. The plates were incubated for 30 min and washed as previously. Alternatively, polyclonal antibodies raised against a mixture of said fusion proteins can be used. The substrate (ABTS, Kirkegaard-Perry) was added (100 μl) to each well. After 15 minutes of incubation the reaction was stopped with 50 μl of 1 % SDS in distilled water, and the absorbances were read at 414 nm (Multiscan, Labsystems). The concentrations of glove extract Hev b6.02, Hev b5, Hev b3 and Hev b1 were read from the appropriate standard curves.

### EXAMPLE 14

### Comparison of Reactivity of Latex Allergic Patient and Control Sera on Microtiter Plates Coated with Individual Latex Allergens or Their Combinations

Microtiter plates (Maxisorp, Nunc, Roskilde, Denmark) were coated with recombinant fusion proteins MBP-Hev b 1, MBP-Hev b 3, MBP-Hev b 5 and Avidin-Hev b 6.02 at a concentration of 10 μg/ml of each allergen individually or mixed (150 μl/well) in sodium phosphate buffer, pH 6.0. For comparison, microtiter plates were also coated with a field latex extract prepared from latex milk. After an overnight incubation at room temperature, the wells were washed four times with 0.05% Tween-20 (BioRad Laboratories, USA) in PBS. The plates were blocked with 0.5% BSA in sodium phosphate buffer, pH 6.5. After removal of blocking buffer the plates were used for testing.

In the test, 100 μl of assay buffer (50 mM sodium phosphate buffer, pH 7.4, containing 50 mM NaCl, 10 mM EDTA, 0.3% BPLA (Bovine plasma albumin, Roche Diagnostics, Germany), 0.03% Tween-20 and 0.1% Proclin 300 (Supelco, USA) was dispensed in each well, 25 μl of patient serum was added, the plates were incubated with shaking at room temperature for 60 min, washed 4 times with 0.05% Tween-20 in PBS. Monoclonal anti-human IgE labelled with HRP (8510-HRP, Medix Biochemica, Finland) diluted 1:50 in assay buffer was added to each well (100 μl). In the similar manner anti-human IgG-HRP, specifically anti-IgG₄-HRP, when appropriate, could be used to detect specific IgG or IgG₄ antibodies against latex allergens.

After an incubation of 30 min with shaking at room temperature, the wells were washed 4 times with 0.05% Tween-20 in PBS. Substrate (ABTS, Kirkegaard-Perry, USA) was added (100 μl) to develop the reaction for 15 minutes, after which 100 μl of 1% SDS (BDH, England) was added to stop the reaction. Absorbances were measured at 414 nm with a microplate reader (Multiscan, Labsystems, Finland). A reading of 0.100 or greater was regarded as positive.

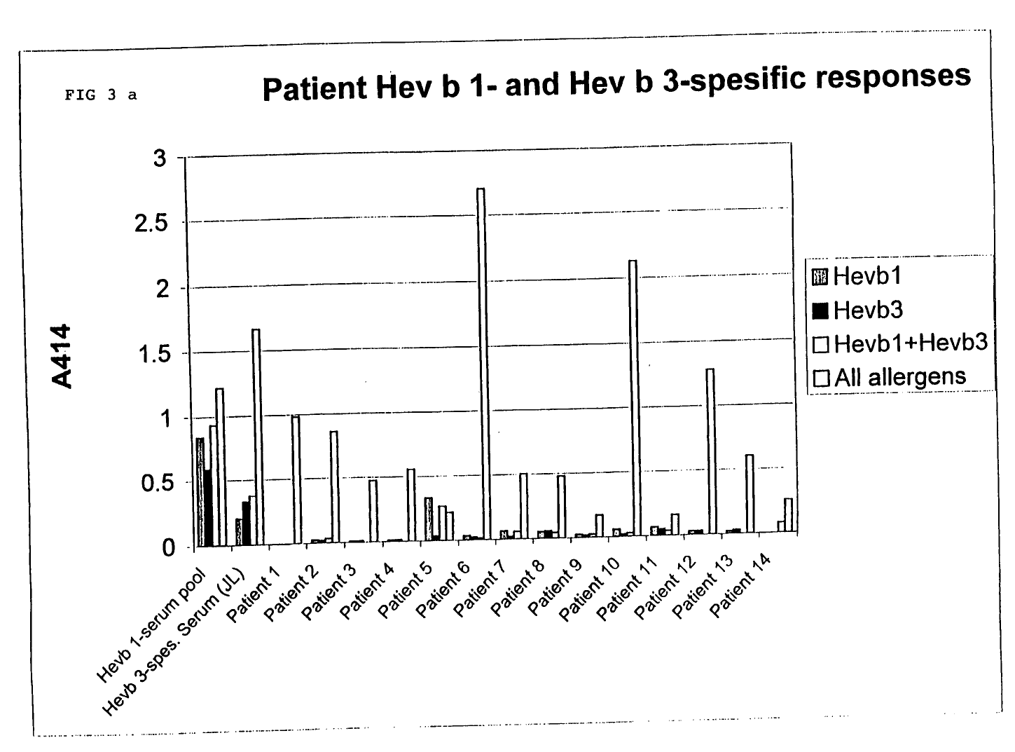

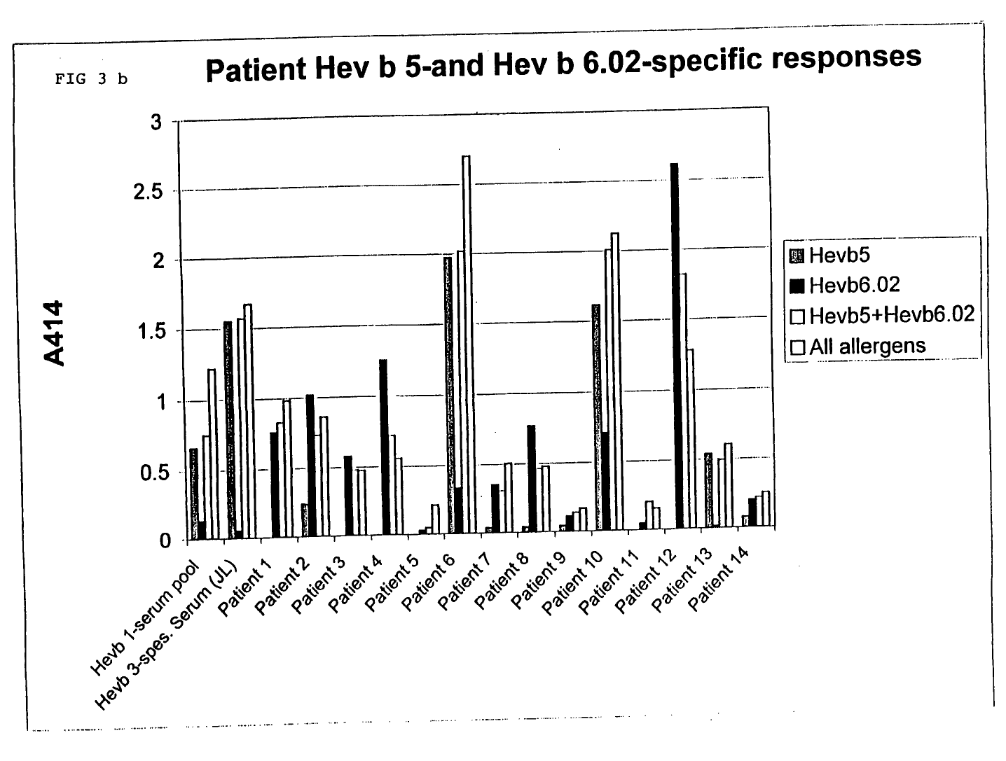

Samples from patients known to be allergic to NRL by skin prick test and patient history and a serum pool from multioperated children were analysed separately, in a combination of two latex allergens and in a combination of four latex allergens. The results are shown in Table 6 and in FIG. 3. Controls run using serum samples of healthy volunteers with no NRL allergy showed no detectable allergen levels.

The method of the invention detected all NRL positive sera when tested on plates coated with the combination of all four allergens, whereas the plated coated with the field latex failed to detect two positive sera. When the results of individual sera from patients allergic to NRL are looked at as the response to one specific latex allergen and a combination of two allergens, a combination of Hev b 5+Hev b 6.02, detected all but one of the positive sera. One could also deduce from the results in Table 6 that combination of Hev b 6.02 and Hev b 1 would suggest 14-15 positive results. Hence the most specific and sensitive combination is the one comprising all four clinically relevant NRL allergens.

TABLE 6
A414 nm (Blanks subtracted)

| | Hev | Hev | Hevb 1 + | All | Field | Hev | Hev b | Hevb 5 + | All |
|---|---|---|---|---|---|---|---|---|---|
| Sample | b 1 | b 3 | Hevb 3 | allergens | latex | b 5 | 6.02 | Hevb 6.02 | allergens |
| Hevb1-serum | 0.838 | 0.589 | 0.933 | 1.217 | 0.925 | 0.657 | 0.131 | 0.750 | 1.217 |
| pool | | | | | | | | | |
| Hevb3-spes. | 0.198 | 0.33 | 0.375 | 1.668 | 0.795 | 1.550 | 0.056 | 1.570 | 1.668 |
| Serum (JL) | | | | | | | | | |
| Patient 1 | <0.01 | <0.01 | <0.01 | 0.985 | 0.448 | <0.01 | 0.76 | 0.830 | 0.985 |
| Patient 2 | 0.024 | 0.019 | 0.035 | 0.863 | 0.361 | 0.239 | 1.019 | 0.733 | 0.863 |
| Patient 3 | 0.010 | 0.011 | <0.01 | 0.473 | 0.144 | <0.01 | 0.575 | 0.479 | 0.473 |
| Patient 4 | 0.011 | 0.013 | <0.01 | 0.556 | 0.078 | <0.01 | 1.252 | 0.72 | 0.556 |
| Patient 5 | 0.329 | 0.036 | 0.264 | 0.213 | 0.504 | <0.01 | 0.029 | 0.047 | 0.213 |
| Patient 6 | 0.034 | 0.022 | 0.013 | 2.706 | 1.017 | 1.971 | 0.335 | 2.018 | 2.706 |
| Patient 7 | 0.062 | 0.021 | 0.057 | 0.504 | 0.218 | 0.037 | 0.353 | 0.309 | 0.504 |
| Patient 8 | 0.051 | 0.057 | 0.044 | 0.479 | 0.478 | 0.037 | 0.768 | 0.464 | 0.479 |
| Patient 9 | 0.023 | 0.018 | 0.023 | 0.168 | 0.053 | 0.042 | 0.109 | 0.137 | 0.168 |
| Patient 10 | 0.055 | 0.018 | 0.026 | 2.126 | 0.654 | 1.612 | 0.710 | 2.008 | 2.126 |
| Patient 11 | 0.064 | 0.050 | 0.034 | 0.157 | 0.541 | <0.01 | 0.047 | 0.202 | 0.157 |
| Patient 12 | 0.027 | 0.027 | <0.01 | 1.278 | 0.12 | <0.01 | 2.616 | 1.817 | 1.278 |
| Patient 13 | 0.019 | 0.027 | <0.01 | 0.605 | 0.217 | 0.537 | 0.014 | 0.492 | 0.605 |
| Patient 14 | nd | nd | 0.077 | 0.253 | 0.156 | 0.076 | 0.200 | 0.22 | 0.253 |
| Control 1 | nd | nd | nd | <0.010 | <0.010 | nd | nd | nd | <0.010 |
| Control 2 | nd | nd | nd | <0.010 | <0.010 | nd | nd | nd | <0.010 |
| Control 3 | nd | nd | nd | <0.010 | <0.010 | nd | nd | nd | <0.010 |

### EXAMPLE 15

### Simultaneous Determination of Specific IgE Antibodies Against NRL Latex Allergens On Plates Coated With MBP-Hev b 1, MBP-Hev b 3, MBP-Hev b 5 and Avidin-Hev b 6.02 Fusion Proteins.

Microtiter plates were coated, with MBP-Hev b 1, MBP-Hev b 3, MBP-Hev b 5 and Avidin-Hev b 6.02 fusion proteins (2 μg/ml of each protein) as described in Example 13.

In the test, 100 μl of assay buffer [50 mM sodium phosphate buffer, pH 7.4, containing 50 mM NaCl, 10 mM EDTA, 0.3% BPLA (Bovine plasma albumin, Roche Diagnostics, Germany), 0.03% Tween-20 and 0.1% Proclin 300 (Supelco, USA)] was pipetted to each well. 25 μl of calibrator or sample was added to each well. After an incubation of 60 minutes on a horizontal shaker at room temperature, the wells were washed four times with PBS-0.05% Tween-20. One hundred microliters of the assay buffer containing 1 μl/ml of anti-human IgE (Clone 8510, Medix Biochemica, Kauniainen, Finland) was added to each well. After an incubation of 30 minutes on a horizontal shaker at room temperature, the wells were washed again. Then 100 μl of HRP-labelled rabbit anti-mouse antibodies (1:1000, P0260, Dako) was added to each well and after an incubation of 30 minutes on a horizontal shaker at room temperature, the wells were washed again. The substrate (ABTS, Kirkegaard-Perry Laboratories, 100 μl) was added to each well. After an incubation of 15 minutes on a horizontal shaker at room temperature, the reaction was stopped with 100 μl of 1% SDS. Absorbances were read at 414 nm with a plate reader (Multiscan, Labsystems, Finland).

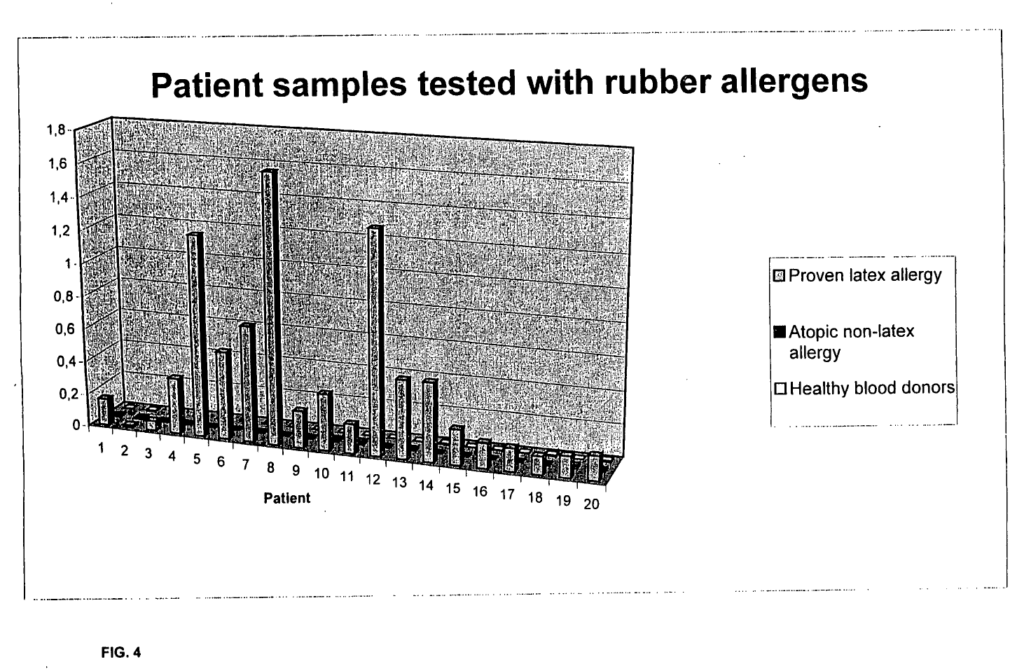

The results obtained with samples from twenty healthy blood donors, twenty skin prick test positive latex allergic patients, and twenty atopic patients without known latex allergy are is given in Table 7 and FIG. 4. A reading of 0.050 greater was regarded as positive.

As shown from the table 7, all healthy blood donors gave a very low absorbance (0.000-0.040), atopic patients gave somewhat higher but still very low readings (0.000-0.014), whereas skin prick test positive latex allergic patient sera gave absorbances varying from 0.060 to 1.624, i.e. all but one (98%) were positive. The clear difference between the different groups tested is clearly illustrated in FIG. 4.

When a total of 100 samples of each test group was tested, in the atopic patient group the mean absorbance was 0.044, in healthy blood donors 0.004 and in latex allergic patient group 0.273 (data not shown).

TABLE 7
Absorbances (at 414 nm), blank subtracted

| Latex allergy positive | Atopic non-latex allergy | Healthy blood donors |
|---|---|---|
| 0.167 | 0.008 | 0.001 |
| 0.088 | 0.010 | 0.005 |
| 0.060 | 0.005 | 0.000 |
| 0.341 | 0.005 | 0.000 |
| 1.223 | 0.001 | 0.000 |
| 0.532 | 0.000 | 0.010 |
| 0.703 | 0.003 | 0.007 |
| 1.624 | 0.002 | 0.001 |
| 0.224 | 0.004 | 0.001 |
| 0.346 | 0.012 | 0.000 |
| 0.178 | 0.014 | 0.000 |
| 1.346 | 0.001 | 0.000 |
| 0.480 | 0.007 | 0.067 |
| 0.482 | 0.000 | 0.005 |
| 0.217 | 0.001 | 0.001 |
| 0.155 | 0.000 | 0.002 |
| 0.143 | 0.002 | 0.002 |
| 0.112 | 0.004 | 0.007 |
| 0.132 | 0.014 | 0.000 |
| 0.151 | 0.007 | 0.009 |

### EXAMPLE 16

### Rapid Immunochromatographic Test for Rubber Allergen Specific IgE Antibody in a Patient's Serum

Membrane cards containing a 25 mm wide SRHF membrane, were cut to about 7 mm wide strips, coated with narrow lines of MBP-Hev b5 fusion protein (0.6 mg/ml), natural hevein (1 mg/ml) and anti-mouse antibody (Dako, Z109, diluted to 2 mg/ml in 100 mM MOPS pH 6.5). After coating the membranes were kept for 10 min in +45° C. and desiccated until use. Glass fiber pad (grade 8980) was soaked in a gold colloid [monoclonal anti-IgE, clone 8510, Medix Biochemica] coated by British Biocell by standard procedures to OD10. After soaking the pad was dried for 1 hr at +35° C. and kept dry until use.

Cellulosic paper (Gelman, type 133) filters were used as an absorbent pad as such and cotton fiber (grade 222) was pretreated with 100 mM MOPS buffer, pH 6.5 containing 2% sucrose (BDH) and 3% Tween 20 (BioRad) by immersing the cotton fiber in the solution for a few seconds, and then dried overnight at room temperature.

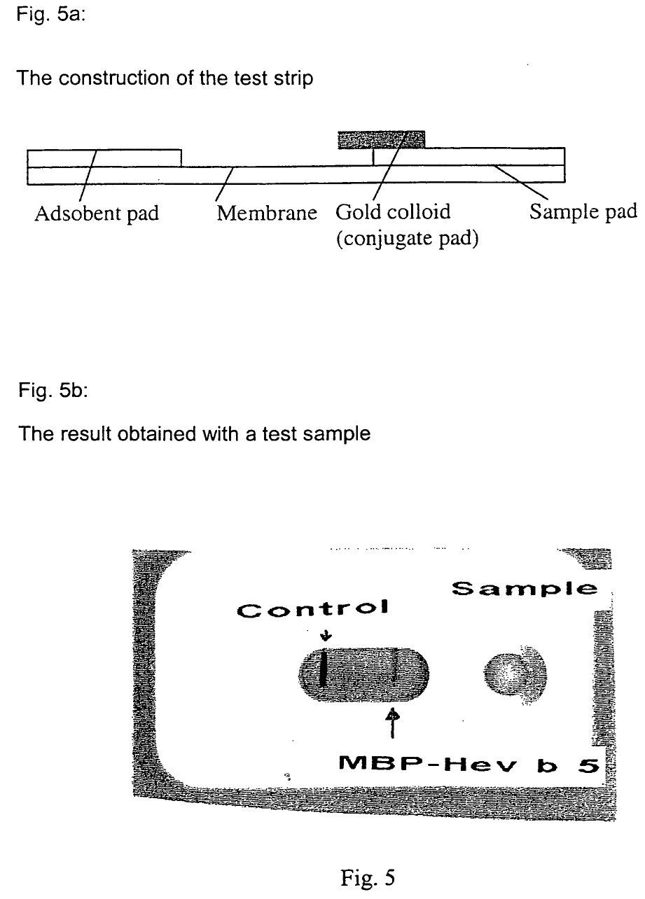

The test was constructed by applying the membrane and filter layers as indicated in FIG. 5. in a plastic cassette (64×36 mm) manufactured by Labplast International, UK.

A sample (100 μl of serum from a patient with known rubber allergy diluted with 150 μl of 50 mM sodium phosphate, 50 mM NaCl, 10 mM EDTA, 0.5% BPLA (bovine plasma albumin, Roche Diagnostics, Germany), 0.03% Tween 20 pH 7.4) was applied to the sample well little by little. After two minutes two red lines were formed, the first line indicating IgE antibodies against Hev b 5 and the second (control) line confirming the performance of the gold colloid.

A serum sample not containing specific IgE antibodies against rubber latex was also run as a control under the same conditions, and no specific line was formed. The appearance of the control line proved that the test was performed properly.

### EXAMPLE 17

### Construction of a Qualitative/Semiquantitative Rapid Test for Latex Allergen Specific IgE

A solution containing recombinant Hev b 6.02, Hev b 1, Hev b 3 and Hev b 5 in 50 mM phosphate buffer pH 7.0 is dispensed as a narrow line on a Immunochromatographic nitrocellulose membrane (for instance Millipore). The membrane is dried at +45° C. for one hour and stored dry until use. The concentration of Hev b 6.02 is defined to give a positive result (a blue line), if the amount of Hev b 6.02, Hev b 1, Hev b 3 and Hev b 5 specific IgE antibodies exceeds the predetermined detection level. The sample pad is impregnated in a solution containing 1-3% Tween-20, 0.2% sucrose and buffer salts. The absorbent pad is normal chromatography paper (for example Whatman 3MM).

Monoclonal anti-IgE antibodies (for example clone 8510, Medix Biochemica, Finland) are coated on blue polystyrene particles (for instance Seradyn particles (0.1-0.3 μm) and dispensed on a conjugate pad and dried.

The sample end contains a sample pad. Blue polystyrene particles are dried on the other end of this pad, which is in contact with the nitrocellulose membrane to allow liquid flow along the membrane by capillary forces. The membrane is in contact with the absorbent pad from the other end. The absorbent pad draws the liquid from the membrane until no liquid is available.

When the sample suspected to contain IgE antibodies against Hev b 6.02 is brought in contact with the test device, it is absorbed by the sample pad and the liquid releases dried blue polystyrene particles from the conjugate pad and flows long the membrane. If the sample contains sufficient amounts of IgE antibodies against Hev b 6.02, Hev b 1, Hev b 3 and/or Hev b 5, a blue line is formed on the membrane indicating that the concentration of latex allergen specific IgE is above the specified threshold value for a positive result.

### EXAMPLE 18

### Construction of a Qualitative/Semiquantitative Rapid Flow Test for Screening Latex Allergens

A solution containing monoclonal anti-Hev b 6.02 antibodies (clone Hb6-2) in 50 mM phosphate buffer, pH 7.0, is dispensed as a narrow line on a nitrocellulose membrane. The membrane is dried at +45° C. for one hour and stored dry until use. The concentration of the antibody is defined to give a positive result (a blue line), if the amount of Hev b 6.02 in the sample exceeds, e.g.,10 μg/l. The sample pad is impregnated in a solution containing 1-3% Tween-20, 0-2% sucrose and buffers salts. The absorbent pad is normal chromatography paper (for example Whatman 3MM).

Monoclonal anti-Hev b 6.02 antibodies against a different epitope than Hb6-2 are coated on blue polystyrene particles (for instance Seradynparticles, 0.1-0.3 μm) and dispensed on a conjugate pad and dried.

The sample end contains a sample pad. Blue polystyrene particles are dried on the other end of this pad, which is in contact with the nitrocellulose membrane to allow liquid flow along the membrane by capillary forces. The membrane is in contact with the absorbent pad from the other end. The absorbent pad draws the liquid from the membrane until no liquid is available.

When the sample suspected to contain latex allergens is brought in contact with the test device, it is absorbed by the sample pad and the liquid releases dried blue polystyrene particles from the conjugate pad and flows along the membrane. If the sample contains Hev b 6.02, a blue line is formed on the membrane indicating a concentration of Hev b 6.02, which is above the specified threshold value (for example 10 μg/l).

### EXAMPLE 19

### Construction of a Qualitative/Semiquantitative Rapid Flow Test for Screening Latex Allergens

Four separate lines of monoclonal antibodies against Hev b6.02. Hev b1, Hev b3 and Hev b5, respectively, are dispensed on a nitrocellulose membrane. The membrane is dried at +45° C. for one hour and stored dry until use. The concentration of each antibody is defined to give a positive result (each line with a different color), if the amount of each latex allergen exceeds the cut off values that have been determined quantitatively. Monoclonal antibodies against different epitopes than the ones used for the dispensed lines (anti- Hev b 6.02, anti-Hev b1, anti-Hev b3 and anti-Hev b5) on the nitrocellulose membrane, are coated on blue polystyrene particles, each of different colour-blue for anti-Hev b 6.02, red for anti-Hev b1, green for anti-Hev b3 and black for anti-Hev b5 (for instance Seradyn-particles, 0.1-0.3 μm) and dispensed on a conjugate pad and dried.

The sample end of the device contains a sample pad. Coloured polystyrene particles are dried on the other end of this pad, which is in contact with the nitrocellulose membrane to allow liquid flow along the membrane by capillary forces. The membrane is in contact with the absorbent pad from the other end. The absorbent pad draws the liquid from the membrane until no liquid is available.

When the sample suspected to contain latex allergens is brought in contact with the test device, it is absorbed by the sample pad and the liquid releases dried colored polystyrene particles from the conjugate pad and flows along the membrane. If the sample contains Hev b 6.02, a blue line is formed on the membrane indicating a concentration of Hev b 6.02, which is above the specified threshold value (for example 10 μg/l). If the sample contains Hev b1, a red line is formed, if Hev b3 is present in the sample, a green line is formed and finally if Hev b5 is present in the sample a black line is formed, each line having the specified cut-off value. If no lines appear, the sample does not contain any of these allergensn at a concentration exceeding the specified threshold value. If all lines are formed, the sample contains all latex allergens at a concentration which is above the specified threshold value.

### EXAMPLE 20

### A Turbidometric Assay

Two monoclonal anti-Hev b 6.02 antibodies, each recognising different epitopes, were simultaneously coated on white polystyrene particles (about 20 nm) in 10 mM phosphate buffer pH 6.5. After coating the particles were washed and blocked according the particle manufacturer's instructions. Finally the particles were suspendended as a 1% solution in the assay buffer (50 mM Tris buffer, pH 7.5, containing 0.03% Tween 20 and 0.3% BSA). In the same way, two monoclonal anti-Hev b5 antibodies, also recognising different epitopes, were coated on polystyrene particles and treated accordingly.

For a turbidometric assay, the particle suspensions were diluted 1:1000 and mixed in equal volumes (suspension A).

Recombinant Hev b6.02 and Hev b5 standards (0 and 0.5-200 μg/l) or latex glove extracts were diluted with the assay buffer to appropriate dilution in order to be measured in the linear standard curve.

Twenty five microliters of the standard or the sample to be assayed are added to appropriate wells in a microtiter plate (Nunc) followed by the addition of 100 μl of diluted antibody coated particle suspension (suspension A). After mixing the turbidity in the wells is measured with an ELISA reader at 340 nm. The standard curve is plotted against absorbance and the concentration of each sample is calculated from the standard curve.

1. A homologous or heterologous immunological method for demonstrating and quantifying simultaneously and/or qualitatively/semiquantitatively demonstrating simultaneously at least two specific latex allergens having clinical relevance in a manufactured rubber product comprising
(a) contacting a sample obtained from said rubber product with a mixture of primary antibodies, and
(b) detecting the bound allergens by a mixture of secondary antibodies, characterized in that said mixtures of primary and/or secondary antibodies each comprise at least two (mono)specific polyclonal or monoclonal antibodies, which are directed against at least two different specific latex allergens having clinical relevance or their functionally equivalent breakdown fragments, sub-units or aggregates or their fusion proteins with a suitable partner, or against specific epitopes thereof, wherein said primary and secondary antibodies have different epitope specificities, said primary antibodies being optionally bound to a solid phase and said secondary antibodies being optionally suitably labelled and/or optionally being bound to a solid phase.

2. A homologous or heterologous immunological method for demonstrating and quantifying simultaneously and/or qualitatively/semiquantitatively demonstrating simultaneously at least two specific latex allergens having clinical relevance from a manufactured rubber product comprising
(a) contacting a sample obtained from said rubber product with a mixture of primary antibodies, and
(b) detecting the bound allergens by secondary antibodies, characterized in that said mixture of primary antibodies comprises at least two (mono)specific polyclonal or monoclonal antibodies, which are directed against at least two different specific latex allergens having clinical relevance or their functionally equivalent breakdown fragments, sub-units or aggregates or their fusion proteins with a suitable partner, or against specific epitopes thereof, and said secondary antibodies comprise polyclonal antibodies raised against a mixture of at least two latex allergens having clinical relevance or their functionally equivalent breakdown fragments, sub-units or aggregates or their fusion proteins with suitable partners, said primary antibodies being optionally bound to a solid phase and said secondary antibodies being optionally suitably labelled and/or optionally being bound to a solid phase.

3. A homologous or heterologous immunological method for demonstrating and quantifying simultaneously and/or qualitatively/semiquantitatively demonstrating simultaneously at least two specific latex allergens having clinical relevance from a manufactured rubber product comprising
(a) contacting a sample obtained from said rubber product with a mixture of primary and secondary antibodies, and
(b) detecting the bound allergens by a suitable detection means, such as colorimetry, turbidometry, nephelometry or fluorometry, characterized in that said mixture of primary and secondary antibodies comprises at least two (mono)specific polyclonal or monoclonal primary antibodies, which are directed against at least two different clinically relevant latex allergens or their functionally equivalent breakdown fragments, sub-units or aggregates or their fusion proteins with a suitable partner, or against specific epitopes thereof, wherein said primary and secondary antibodies have different epitope specificities, said primary and secondary antibodies being bound to a solid phase and optionally being suitably labelled for detection.

4. An immunochemical method for detecting simultaneously the presence or absence of specific IgE antibodies against at least two latex allergens of clinical relevance, characterized by contacting a specimen, which is obtained from a patient suspected of suffering from latex allergy, with a mixture of at least two clinically relevant latex allergens and detecting the immunological complex thus formed.

5. An immunochemical method for detecting simultaneously the presence or absence of specific IgG4 antibodies against at least two latex allergens of clinical relevance, characterized by contacting a specimen, which is obtained from a patient suspected of suffering from latex allergy, with a mixture of clinically relevant latex allergens, and detecting the immunological complex thus formed.

6. A screening method for the simultaneous detection of the presence or absence of specific IgE and/or IgG4 against at least two latex allergens of clinical relevance, characterized by contacting a specimen, which is obtained from a patient suspected of suffering from latex allergy, with a mixture of at least two clinically relevant latex allergens and detecting the immunological complex thus formed.

7. An immunological method according to any one of claims 1 to 6, characterised in that said clinically relevant latex allergens are chosen from any natural rubber latex allergens which retain their allergenic properties through the manufacturing processes and are detectable in finished products made of or containing natural rubber latex.

8. An immunological method of any one of claims 1 to 7, characterized in that said clinically relevant latex allergens are chosen from at least two of the following: rubber elongation factor (REF or Hev b 1), rubber particle associated protein (Hev b3), acidic 16 kD protein (Hev b5) and hevein (Hev b6.02) in a natural or recombinant form.

9. An immunological method of any one of claims 1 to 7, characterized in that the clinically relevant latex allergens are chosen from natural or recombinant Hev b 6.02 and any or all of natural or recombinant Hev b 1, Hev b 3 and Hev b 5 or fusion proteins thereof with a suitable fusion partner or functionally equivalent fragments or aggregates thereof.

10. An immunological method of any one of claims 1 to 7, characterized in that the clinically relevant latex allergens are at least natural or recombinant Hev b 6.02 and Hev b 5 or fusion proteins thereof with a suitable fusion partner or functionally equivalent fragments or aggregates thereof.

11. An immunological method of any one of claims 1 to 7, characterized in that the clinically relevant latex allergens are at least natural or recombinant Hev b 6.02 and Hev b 1 or fusion proteins thereof with a suitable fusion partner or functionally equivalent fragments or aggregates thereof.

12. An immunological method of any one of claims 1 to 7, characterized in that the clinically relevant latex allergens are natural or recombinant Hev b 1, Hev b 3, Hev b 5 and Hev b 6.02 or fusion proteins thereof with a suitable fusion partner or functionally equivalent fragments or aggregates thereof.

13. An immunological method according to any one of claims 1 to 3, characterised in that the primary and/or secondary antibodies are monoclonal antibodies.

14. An immunological method according to any one of claims 1 to 3, characterised in that the primary antibodies are monoclonal antibodies and secondary antibodies are polyclonal antibodies.

15. An immunological method according to any one of claims 1 to 3, characterised in that the primary antibodies are bound to a solid phase and the secondary antibodies are suitably labelled.

16. An immunological method according to claim 3, characterised in that the primary and secondary antibodies are bound to a solid phase and turbidometry is used for the detection.

17. A test kit for simultaneously detecting the presence or absence of specific IgE and/or IgG₄ antibodies against at least two latex allergens of clinical relevance, characterized by containing reagents the reagents needed for performing the immunological methods of claims 4 to 5.

18. A test kit for demonstrating simultaneously and quantifying and/or qualitatively/semiquantitatively demonstrating simultaneously at least two specific latex allergens having clinical relevance from manufactured rubber products, characterised by comprising
(1) a mixture of at least two primary antibodies, the antibodies being (mono)specific polyclonal or monoclonal antibodies, which are directed against different specific latex allergens having clinical relevance or their functionally equivalent breakdown fragments, sub-units or aggregates or their fusion proteins with a suitable partner, or specific epitopes thereof, said antibodies being optionally bound to a solid phase,
(2a) a mixture of at least two secondary antibodies, the antibodies being (mono)specific polyclonal or monoclonal antibodies, which are directed against other specific latex allergens having clinical relevance or their functionally equivalent breakdown fragments, sub-units or aggregates or their fusion proteins with a suitable partner, or against another epitopes thereof, said antibodies being optionally suitably labelled and/or bound to a solid phase, or
(2b) polyclonal antibodies raised against a mixture of at least two specific latex allergens having clinical relevance or their functionally equivalent breakdown fragments, sub-units or aggregates or their fusion proteins with suitable partners, said antibodies being optionally suitably labelled and/or bound to a solid phase,
(3) a reagent or reagents needed for the detection, and
(4) the standards needed for the quantification of the specific latex allergens having clinical relevance.

19. A test kit according to claim 18, characterised by further comprising
(5) a reagent or reagents for the elution of the latex allergens having clinical relevance from a sample suspected to contain such allergens.

20. A test kit according to claim 18 or 19, characterised in that the two clinically relevant latex allergens are any natural rubber latex allergens which retain their allergenic properties through the manufacturing processes and are detectable in finished products made of or containing natural rubber latex.

21. A composition containing simultaneously predetermined amounts of natural or recombinant natural rubber latex allergens of clinical relevance or fusion proteins thereof with a suitable fusion partner or functionally equivalent fragments or aggregates thereof in a suitable formulation.

22. A composition of claim 21, characterized by containing natural or recombinant Hev b 6.02 and any or all of recombinant Hev b 1, Hev b 3 and Hev b 5 or fusion proteins thereof with a suitable fusion partner or functionally equivalent fragments or aggregates thereof.

23. A composition of claim 21, characterized by containing natural recombinant Hev b 1, Hev b 3, Hev b 5 and Hev b 6.02 or fusion proteins thereof with a suitable fusion partner or functionally equivalent fragments or aggregates thereof.

24. The simultaneous use of natural or recombinant Hev b 1, Hev b 3, Hev b 5 and Hev b 6.02 or fusion proteins thereof in the diagnosis of NRL allergy.

25. The use of natural or recombinant Hev b 1, Hev b 3, Hev b 5 and Hev b 6.02 or fusion proteins thereof in the simultaneous detection the possible residual content of allergenic proteins or peptides derived from natural rubber latex in a finished product made of or containing natural rubber latex.
